(19) United States
(12) Patent Application Publication
(10) Pub. No.: US 2008/0299109 A1
(43) Pub. Date: Dec. 4, 2008

(54) MECHANISM OF ASTRICYTE-NEURON SIGNALING

(75) Inventors: Changjoon Justin LEE, Seoul (KR); Dong-Ho Woo, Seoul (KR); Stephen F. Traynelis, Atlanta, GA (US)

Correspondence Address: LEXYOUME IP GROUP, LLC, 5180 PARKSTONE DRIVE, SUITE 175, CHANTILLY, VA, 20151, US

(73) Assignee: Korea Institute of Science and Technology, Seoul (KR)

(21) Appl. No.: 12/022,510

(22) Filed: Jan. 30, 2008

(30) Foreign Application Priority Data
May 31, 2007 (KR) 10-2007-0053375

(51) Int. Cl. A61K 38/48; C12Q 1/02; A61P 25/00

(52) U.S. Cl. 424/94.64; 435/29

(57) ABSTRACT

The present invention relates to a novel communication mechanism between astrocytes and neurons at a synapse. More specifically, the present invention relates to a signaling mechanism between astrocytes and neurons, by activating astrocytic G-protein coupled receptors, thereby activating glutamate receptors on a membrane of neighboring postsynaptic neurons, resulting in increasing the level of intracellular Ca²⁺ and inducing a depolarization inward current to control neurotransmission in neurons.

### CROSS-REFERENCE TO RELATED APPLICATION

This application claims priority to and the benefit of Korean Patent Application No. 10-2007-0053375 filed in the Korean Intellectual Property Office on May 31, 2007, the entire contents of which are incorporated herein by reference.

### BACKGROUND OF THE INVENTION

(a) Field of the Invention

The present invention relates to a novel communication mechanism between astrocytes and neurons at a synapse. More specifically, the present invention relates to a signaling mechanism between astrocytes and neurons, by activating astrocytic G-protein coupled receptors, thereby activating glutamate receptors on a membrane of a neighboring postsynaptic neuron, resulting in increasing the level of intracellular Ca²⁺ and inducing a depolarization inward current to control neurotransmission in neurons.

(b) Description of the Related Art

Astrocytes play important roles in maintaining normal activities of the brain as well as in developing the brain. It has been accepted for the past several decades that astrocytes in the brain merely have some functions of properly controlling neurotransmitters secreted from neurons, or assisting neuron activities by controlling ion concentration in the brain. Recently, astrocytes have been known to exhibit the functions of synaptic formation, control of the number of synapses, synaptic plasticity, and the like, and to participate in the development from neural stem cells to neurons.

However, there have been almost no studies on active functions of astrocytes, only on passive functions to aid neural functions. In particular, the fact that astrocytes actively function in signal transduction between neurons and the mechanism of how the astrocytes function have not been reported.

### SUMMARY OF THE INVENTION

The present invention reveals a signal transduction pathway between neurons and astrocytes and the roles of astrocytes in the pathway.

An embodiment of the present invention provides a technique of controlling neurotransmission at an adjacent neuron by operating astrocytes.

Another embodiment of the present invention provides a screening method of a treatment agent for neurological diseases by using the neurotransmission mechanism between neurons and astrocytes.

### BRIEF DESCRIPTION OF THE DRAWINGS

FIG. 1a shows a superimposed ratio image of Fura2-AM loaded cultured wild-type mouse astrocytes before and after TFLLR application, and

FIG. 1b shows representative traces of ratio amplitude changes in Fura-2 fluorescence ratio by pressure application of a brief pulse of TFLLR, bradykinin, 2-methyl-thio-ATP, and ATP.

FIG. 2a shows ratio images of control and Fura2-AM loaded cultured wild-type mouse astrocytes before and after thrombin application (left two panels), and after TFLLR application on wild-type and PAR1−/− mouse astrocytes (right two panels), and

FIG. 2b shows superimposed representative ratio response time courses under various conditions.

FIG. 3a shows a DIC image of a glial cell (left panel), and results obtained by applying 10 mV voltage steps to this cell under voltage clamp,

FIG. 3b shows the changes in fluorescent intensity in the glial cell during application of TFLLR,

FIG. 3c shows fluorescent intensity in a glial cell (left), and fluorescent change as a ΔF/Fo before (center, Baseline) and after TFLLR application (right),

FIG. 3d shows the average changes in fluorescence (±SEM),

FIG. 3e shows a DIC image of a CA1 pyramidal neuron (left panel), and membrane voltage change and action potential in the neuron (right panel), and

FIG. 3f shows the changes in somatic fluorescent intensity in dye-loaded CA1 neurons.

FIG. 4 shows that PAR1 activation stimulates Ca²⁺-dependent release of glutamate in astrocytes.

FIG. 5a is a schematic illustrating experimental setup and a GFP fluorescent image of astrocyte—GluR1(L497Y)/GFP transfected HEK cell co-culture (upper left panel), and ratio images depending on pressure-applied TFLLR (lower left and right panels),

FIG. 5b shows the results of the quantification of the fluorescence increase in response to brief (<1 sec) pressure application of TFLLR, ATP, and bradykinin in a wild-type astrocyte (upper trace), and the inward current induced in an adjacent GluR1(L497Y)-transfected HEK cell (lower trace),

FIG. 5c shows Fura2 fluorescence ratio (upper trace) and inward current (lower trace) in a GluR1(L497Y)-transfected HEK cell,

FIG. 5d shows the current amplitude changes in GluR1(L497Y) transfected HEK cells with TFLLR and/or CNQX application to wild-type and PAR1−/− astrocyte cultures,

FIG. 5e shows the dose response relationship and current response to pressure application of TFLLR converted to concentration using the dose response relationship and maximal current response of the GluR1(L497Y) transfected HEK cell as described in the following Formula I,

FIG. 5f shows the concentration responses from 7 cells superimposed (upper panel) and the average thereof (lower panel), and

FIG. 5g summarizes the glutamate evoked current response (%) and the peak concentration in GluR1(L497Y) transfected HEK cells to TFLLR application to wild-type and PAR1−/− astrocytes.

FIG. 6a shows images of GFAP-GFP labeled astrocytes (green) plated onto GluR1(L497Y) transfected HEK cells (red) (upper panel), and a DIC image of the recording electrode and pressurized agonist filled pipette in the co-culture (lower panel), and

FIG. 6b shows representative traces of Fura-2 fluorescence increase in a GFAP-GFP labeled astrocyte (upper trace), the inward current from a GluR1(L497Y) transfected HEK cell (lower trace), and the response to 10 s application of a maximally effective concentration of glutamate on the same cell (insert).

FIG. 7 shows the glutamate release from neurons measured using GluR1(L497Y)-transfected HEK cells (a), the glutamate-induced current amplitude change (b), and the response to application of a maximally effective concentration of glutamate on GluR1(L497Y)-transfected HEK cells (c).

FIG. 8a shows a photomicrograph of a PAR1−/− cortical neuron loaded with Oregon Green BAPTA2,

FIG. 8b shows a fluorescent image of the same PAR1−/− neuron loaded with Oregon Green 488 BAPTA-2 (450-490 nm excitation; 520 nm emission),

FIG. 8c shows the change in the level of Ca²⁺ by PAR1 in astrocytes, and

FIG. 8c is a graph showing that PAR1 activation in astrocytes induces APV-sensitive inward current in PAR1−/− neurons.

FIG. 9a shows a representative trace showing thrombin-induced inward current,

FIG. 9b shows a summary of amplitude changes of inward current induced by thrombin and TFLLR with and without co-application of APV,

FIG. 9c shows the membrane current variance with application of thrombin,

FIG. 9d shows the membrane current variances measured from CA1 pyramidal cells held under voltage clamp (−60 mV) by thrombin and the PAR1 agonist peptide TFLLR,

FIG. 9e shows current clamp recordings from a CA1 pyramidal cell (left panel) showing depolarization and spike-firing during application of thrombin (1.5 mM Mg²⁺) (left panel), and showing a significant depolarization of the membrane potential (right panel) from 22 neurons, and

FIG. 9f shows the decrease of depolarization by APV application.

FIG. 10a shows the current voltage (I-V) relationship for evoked NMDA EPSCs recorded at 5 min intervals from CA1 pyramidal cells under voltage clamp,

FIG. 10b shows a peak current by plotting as a function of membrane potential from a CA1 pyramidal cell before and 12.5 min following treatment with thrombin,

FIG. 10c shows the EPSCs recordings evoked from the case of being blocked by the competitive NMDA receptor antagonist D-APV, and

FIGS. 10d and 10e shows I-V curves of control and thrombin treated cases.

FIG. 11a shows recordings showing the different rise times of mEPSCs recorded under voltage clamp from CA1 pyramidal cells,

FIG. 11b shows recordings showing the rise times of mEPSCs from II cells for 5 min,

FIG. 11c shows recordings showing the fast rising mEPSCs in the absence and presence of TFLLR,

FIG. 11d is a bar graph showing the decay time constant τ₁ (left panel) and amplitude (right panel) of fast rising mEPSCs recorded under control conditions or during application of TFLLR or APV,

FIG. 11e shows superimposed normalized average traces showing slow rising mEPSCs in the absence and presence of TFLLR,

FIG. 11f is a bar graph showing the decay time constant τ₁ (left panel) and amplitude (right panel) of the fastest component of mEPSCs recorded under all conditions,

FIG. 11g shows superimposed normalized average traces showing slow rising mEPSCs possessing a slow NMDA receptor mediated component, and

FIG. 11h is a graph showing the time constants (left panel) and amplitudes (right panel) of the two synaptic components depending on TFLLR and Mg²⁺ conditions.

FIG. 12a shows average EPSP obtained from a rat CA1 pyramidal cell before (blue) and during application of the PAR1 agonist TFLLR (black) (left panel), and the difference potential between the EPSP recorded under control and APV, or following TFLLR and APV (right panel),

FIG. 12b shows the time course of the peak amplitude during application of TFLLR (left panel) and the average amplitude of the EPSP (right panel),

FIG. 12c shows the time course of the area under the EPSP during application of TFLLR (left panel), and the average area (right panel), and

FIGS. 12d and 12e show graphs showing the potentiation of synaptic NMDA component of EPSPs by PAR1 activation near the physiologic temperature (34° C.).

FIG. 13a is a diagram illustrating the mechanism of how PAR1 activation in astrocytes subsequently leads to potentiation of synaptic NMDA receptor function secondary to glutamate mediated spine head depolarization and reduction in Mg²⁺ block of synaptic NMDA receptors, and

FIG. 13b is a diagram illustrating the mechanism of how sustained release of glutamate from astrocytes following PAR1 activation could lead to tonic activation of perisynaptic NMDA receptors.

FIG. 14a shows representative traces of a TFLLR-induced fluorescence increase in a wild-type astrocyte (upper trace) recorded together with the inward current from a GluR1(L497Y) transfected HEK cell (lower trace), which are co-cultured in a glutamine-free medium, and

FIG. 14b shows the amplitude changes by TFLLR and CNQX in the glutamine-free medium.

FIG. 15a shows the record for the current changes in an NR1/NR2A transfected HEK 293 cell under a gramicidin-D perforated patch, voltage clamp configuration (left panel), and a bar graph showing the maximum glutamate-induced currents from 5 cells before and after being treated with thrombin,

FIG. 15b shows an I-V relationship obtained by applying voltage ramps from +100 mV to −100 mV and subtracting the traces before and during glutamate application on different HEK 293 cells, and

FIG. 15c shows an image of Fura2 fluorescence intensity on HEK cells expressing NR1/NR2A (left panel), and a graph showing the relative fluorescence amplitude (right panel).

### DETAILED DESCRIPTION OF THE EMBODIMENTS

In the following detailed description, only certain exemplary embodiments of the present invention have been shown and described, simply by way of illustration.

As those skilled in the art would realize, the described embodiments may be modified in various ways, all without departing from the spirit or scope of the present invention.

Accordingly, the drawings and description are to be regarded as illustrative in nature and not restrictive.

Like reference numerals designate like elements throughout the specification.

In addition, unless explicitly described to the contrary, the word “comprise”, and variations such as “comprises” or “comprising”, will be understood to imply the inclusion of stated elements, but not the exclusion of any other elements.

The present invention relates to a novel communication mechanism between astrocytes and neurons at a synapse. More specifically, the present invention relates to a signaling mechanism between astrocytes and neurons, by activating astrocytic G-protein coupled receptors, thereby activating glutamate receptors on a membrane of a neighboring postsynaptic neuron, resulting in increasing the level of intracellular Ca²⁺ and inducing a depolarization inward current, to control neurotransmission in neurons.

Astrocytes express a wide range of G-protein coupled receptors that trigger release of intracellular Ca²⁺, including P2Y, bradykinin, protease activated receptors (PARs), and the like. By using the highly sensitive sniffer-patch technique (T. G. J. Allen, Trends Neurosci., Vol. 20, No. 5 pp. 192-107, 1997; the entire contents of which are incorporated herein by reference), the present inventors demonstrate that the activation of P2Y receptors, bradykinin receptors, and protease activated receptors all stimulate glutamate release from cultured or acutely dissociated astrocytes. Based on this matter, the present inventors reveal the signal transduction pathway between neurons and astrocytes and the mechanism of controlling neurotransmission of neurons by astrocytes, to complete the present invention. Such pathways and mechanisms are found in rodents as well as human beings, and moreover, may be widely applied to all mammals. The astrocytes involved in the pathways and mechanisms may be any astrocytes present in all nerve tissues, and preferably any astrocytes present in all brain tissues, for example, any astrocytes present in the hippocampus C1 domain, cortex, striatum, and the like, are but not limited thereto.

The G-protein coupled receptor in the present invention may include all known G-protein coupled receptors, for example, selected from the group consisting of P2Y receptors, bradykinin receptors, protease activated receptors (PARs), and the like. Preferably, PARs are found to be expressed in a great amount specifically in astrocytes compared with other nerve tissues. Therefore, in a preferable embodiment, the G-protein coupled receptor may be PAR(s). Furthermore, of the above receptors, PAR1 which is one of PARs may be utilized as a model system because of favorable pharmacological and molecular tools, its prominent expression in astrocytes, as well as its high relevance to neuropathological processes, but is not limited thereto.

An embodiment of the present invention relates to a mechanism of astrocyte-neuron signal transduction, wherein:

G-protein coupled receptor(s) on astrocyte(s) is (are) activated;

intracellular Ca²⁺ concentration in the astrocyte(s) is increased by the activation;

glutamate release from the astrocytes is increased by the increased intracellular Ca²⁺;

a glutamate receptor(s) on a membrane of an adjacent postsynaptic neuron (dendrite) is (are) activated by the glutamate released from astrocyte(s);

inward current into the glutamate receptor-activated neuron is increased; and

neural depolarization is induced.

When astrocytes are treated with a PAR1 activator, polypeptide TFLLR, and/or thrombin, the astrocytic intracellular Ca²⁺ level is increased. Therefore, TFLLR and/or thrombin may have a stimulating effect on all events caused by increase in astrocytic intracellular Ca²⁺ by PAR1 activation. Such increase in astrocytic intracellular Ca²⁺ level by the PAR1 activator means that the intracellular Ca²⁺ level is increased by PAR1 activation in astrocytes. As described above, among G-protein coupled receptors, PARs, especially PAR1, may be the most suitable G-protein coupled receptor in the present invention, complying with the fact that the signal transduction pathway is started from astrocytes, since it is expressed in a great amount selectively in astrocytes. The increase in intracellular Ca²⁺ level is found in astrocytes in acutely dissociated brain slices as well as cultured astrocyts (in vitro).

As evidenced in Experimental Example 2 below, when a glutamate receptor activator is added to a neuron, intracellular Ca²⁺ level is increased, whereas when a PAR1 activator is added, no increase in intracellular Ca²⁺ level occurs. That is, PAR1 activation is not general with all brain tissues, but is specific to astrocytes, which is firstly revealed in the present invention.

The glutamate release by activation of G-protein coupled receptors is Ca²⁺-dependent. It has been conventionally accepted that astrocytes relate to homeostasis of glutamate, and play passive roles in absorption and metabolism of synaptically released glutamate through transporters with several molecular characters. However, the present invention firstly reveals the active roles of astrocytes in signal transduction to adjacent neurons and in control of the signal transduction. The Ca²⁺-dependent glutamate release from astrocytes induced by activation of G-protein coupled receptors may occur by any extralcellular releasing mechanisms including channel-related mechanisms, exocytosis, and the like.

The glutamate released from astrocytes to extralcellular space, i.e., synaptic space, activates glutamate receptors, for example the N-methyl-D-aspartic acid (NMDA) receptor (accession no. AAA21180), positioned on adjacent neurons, especially postsynaptic neurons. Glutamate receptors may include two groups, where one is metabotropic glutamate receptors including the mGluR family, and the other is ionotropic glutamate receptors functioning as ligands regulating ion channels as well. In an embodiment of the present invention, the glutamate receptor on an adjacent postsynaptic neuron activated by a G-protein coupled receptor on astrocytes may be selected from ionotropic glutamate receptors, especially NMDA receptors. In the activation of a neural glutamate receptor by an astrocytic G-protein coupled receptor according to the present invention, NMDA receptors have approximately a 100-fold lower EC₅₀ than that of other glutamate receptors, such as AMPA or kinase receptors, indicating that NMDA receptors may be suitably used as the glutamate receptor in the present invention. Such activation of a NMDA receptor by glutamate may be blocked by a competitive NMDA receptor antagonist, D-2-amino-5-phosphono-valeric acid (APV).

In the mechanism of the present invention, an increase in inward current to a neuron and depolarization occur together with such activation of a glutamate receptor on an adjacent postsynaptic neuron. As described above, when a PAR1 activator, thrombin, is treated, the subsequent depolarization process at an adjacent neuron is sensitive to an NMDA receptor antagonist, APV, confirming that the neural depolarization in the mechanism of the present invention is caused by the activation of a neural NMDA receptor by glutamate released from astrocytes. Further, the PAR1 activation in astrocytes decreases synaptic Mg²⁺ blocks by synaptic NMDA receptors, and increases excitatory postsynaptic conductance (EPSCs) during synaptic neurotransmission.

Since neural depolarization by PAR activation occurs when the extracellular Mg²⁺ concentration is maintained at a stable level, the decreases in synaptic Mg²⁺ blocks by synaptic NMDA receptors caused by PAR1 activation may also be considered as a main effect of PAR1 activation. The extracellular Mg²⁺ concentration necessary for effective depolarization in a neuron may be from 0.2 mM to 2 mM.

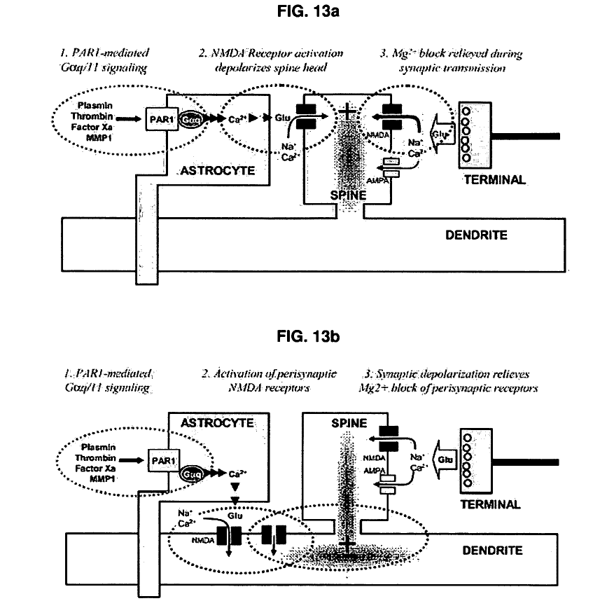

As described above, the present invention may be characterized in that G-proteins coupled receptors, preferably PARs (e.g., PAR1), in astrocytes are capable of triggering the glutamate release from astrocytes in the Ca²⁺-dependent manner, and subsequently, of controlling (activating) the action of postsynaptic neural NMDA receptors, resulting in a Ca²⁺ influx, and to induce depolarization due to the Ca²⁺ influx by the activation of NMDA receptors. The astrocyte-induced depolarization of neurons relieves voltage-dependent Mg²⁺ blocks of synaptic NMDA receptors to potentiate subsequent synaptic NMDA receptor-mediated EPSPs (FIG. 13a).

In the present invention, only ˜0.1 mV somatic depolarization is observed, illustrating the reason that spine depolarization could effectively relieve synaptic Mg²⁺ blocks without causing profound somatic depolarization.

The increase of inward current and depolarization in adjacent postsynaptic neurons (dendrites) caused by PAR activation in astrocytes occurs in dendritic peri-synaptic as well as in the spine head of neurons adjacent to astrocytes (FIG. 13). In addition, the astrocytic PAR1 activation allows enhancing NMDA receptor components of EPSCs in the Mg²⁺-dependent manner, and in particular, of distal but not proximal EPSPs, suggesting that PAR1-induced potentiation of NMDA receptors involves depolarization of the distal dendrites. That is, the present invention firstly reveals that the activation of G-protein coupled receptors, preferably PARs, more preferably PAR1, on astrocytes, specifically functions on postsynaptic neurons but not on presynaptic neurons, to induce the activation of glutamate receptors and depolarization in the same orientation with that of neurotransmission in neurons, suggesting that activation of a G-protein coupled receptor of astrocytes directly functions on postsynaptic neurons in the same orientation with that of neurotransmission.

Therefore, an embodiment of the present invention provides a technique to control the signal transduction mechanism between an astrocyte and a neuron, by controlling G-protein coupled receptor(s) on an astrocyte, thereby controlling the activity of glutamate receptor(s) (e.g., an N-methyl-D-aspartic acid (NMDA) receptor) on the membrane of a postsynaptic neuron by the glutamate released from the astrocyte.

Although glutamate is an important neurotransmitter of the central nervous system, the over-released glutamate functions as a neurotoxin and kills neurons, and thus it is important to maintain the homeostasis of glutamate. Therefore, the mechanism of controlling the glutamate release from astrocyte(s) by astrocytic G-protein coupled receptor(s) (preferably PARs, and more preferably PAR1), and thereby controlling the activity of a glutamate receptor (preferably NMDA receptor) on an adjacent postsynaptic neural membrane, may be used in the following two aspects: in one aspect, when neurotransmission is declined, the mechanism may be used in stimulating neurotransmission and/or improving all glutamate receptor-mediated brain functions by activating astrocytic G-protein coupled receptor(s), and thereby activating glutamate receptor(s) on an adjacent postsynaptic neuron; and in the other aspect, when glutamate is over-released and exhibits neurotoxicity, the mechanism may be used in lowering the neurotoxicity to adjacent postsynaptic neuron and/or treating acute or degenerative brain disease cased by the neurotoxicity of glutamate, by inhibiting the activity of the astrocytic G-protein coupled receptor and over-release of glutamate from astrocytes.

In the signal transduction mechanism between an astrocyte and a neuron according to the present invention, neurotransmission to an adjacent postsynaptic neuron may be induced by activating astrocytic a G-protein coupled receptor(s). Therefore, an embodiment of the present invention relates to a method of stimulating glutamate receptor-mediated neurotransmission, by activating G-protein coupled receptors on astrocytes, that may be one or more selected from the group consisting of P2Y receptors, bradykinin receptors, protease activated receptors (PARs), and the like, and preferably PARs, and more preferably PAR1, to activate glutamate receptors, preferably NMDA receptors, on adjacent postsynaptic neurons. Considering that the process of increase of intracellular Ca²⁺ concentration by activation of astrocytic G-protein coupled receptors and glutamate release is an initial step of stimulating neurotransmission to adjacent postsynaptic neurons, the method of stimulating a glutamate receptor-mediated neurotransmission may have an effect on synaptic plasticity by activating astrocytic G-protein coupled receptors and thereby activating glutamate receptors, preferably NMDA receptors, on adjacent postsynaptic neurons, and may include a method of improving the ability of recognition, perception, motion, memory, and/or learning mediated by the glutamate receptors, preferably NMDA receptors.

In another embodiment, the present invention relates to a composition for glutamate receptor activation on adjacent postsynaptic neurons, containing, as an active ingredient, one or more astrocytic G-protein coupled receptor activators selected from the group consisting of activators for P2Y receptors, bradykinin receptors, protease activated receptors (PARs), and the like, and preferably PAR1. In still another embodiment, the present invention relates to a neurotransmission stimulating agent for stimulating glutamate receptor-mediated neurotransmission, containing one or more astrocytic G-protein coupled receptor activators as described above as an active ingredient. In still another embodiment, the present invention relates to a composition for improving the ability of recognition, perception, motion, memory, and/or learning mediated by the glutamate receptor, preferably an NMDA receptor, containing one or more astrocytic G-protein coupled receptor activators as described above as an active ingredient. Said PAR1 activator may be one or more selected from the group consisting of the polypeptide TFLLR, and thrombin, wherein the effective amount of TFLLR may be from 10 uM to 100 uM, and the effective amount of thrombin may be from 10 nM to 100 nM.

In yet still another embodiment, the present invention relates to a method of screening a glutamate receptor activating agent and/or a glutamate receptor-mediated neurotransmission stimulating agent, including the steps of:

contacting a candidate compound selectively with a G-protein coupled receptor on an astrocyte;

measuring the activation of a glutamate receptor, preferably an N-methyl-D-aspartic acid (NMDA) receptor, on an adjacent postsynaptic neuron; and

determining the candidate compound as a neurotransmission stimulating agent that stimulates a glutamate receptor, preferably an NMDA receptor-mediated neurotransmission agent, when the glutamate receptor in the case of contacting the candidate compound is more activated compared with a case of not contacting the candidate compound. Said glutamate receptor activating agent may be used as a composition for improving the ability of recognition, perception, motion, memory, and/or learning mediated by the glutamate receptor, preferably an NMDA receptor, due to its effect of inducing glutamate release from an astrocyte to activate the glutamate receptor on adjacent postsynaptic neuron.

The activation of a glutamate receptor on an adjacent postsynaptic neuron may be determined by an inward current change through the glutamate receptor on an adjacent postsynaptic neuron before and after treating the candidate compound and/or depolarization, where the activation is confirmed when the inward current change after treating the candidate compound is increased and/or the depolarization is induced. The inward current change and depolarization for confirming the activation of a glutamate receptor may be determined by all conventional manners known to the relevant art, for example a patch clamp and the like. In addition, the step of selectively contacting a candidate compound with a G-protein coupled receptor on an astrocyte may be performed by all conventional manners known to the relevant art. For example, the step may be done by treating the candidate compound after treating antagonists against all receptors on an astrocyte other than the specific G-protein coupled receptor to be examined, thereby inactivating all the astrocytic receptors except the would-be examined receptor, but is not limited thereto.

In another aspect, when an astrocytic G-protein coupled receptor is over-activated and glutamate is over-released from an astrocyte, glutamate receptors on adjacent postsynaptic neurons may be over-activated, and thereby influx of Ca²⁺ ions as well as glutamate is increased, causing neurotoxicity. Such over-release of glutamate from astrocytes and over-activation of glutamate receptors are associated with various acute or degenerative brain diseases including epileptic seizures, glutamate induced excitotoxicity during seizures, ischemia, stroke, cerebral hemorrhage, epilepsy, brain injury (head injury), hypoxia, and the like. In particular, under the condition of dysfunction of the blood-brain barrier, for example by destruction of the blood-brain barrier, thrombin is over-released from blood vessels, allowing activating astrocytic PAR1, inducing glutamate release from astrocytes in a great amount, over-activating neuronal NMDA receptors, and causing neural injury.

The present invention firstly examines the mechanism in which the activation of astrocytic PAR allows activating glutamate receptors, preferably NMDA receptors, on adjacent postsynaptic neurons. The mechanism may be useful in developing neuroprotecting agents for protecting nerve cells from neurotoxicity by over-release of glutamate from astrocytes, and treatment agents for preventing and/or treating one or more glutamate over-release associated diseases selected from the group consisting of epileptic seizures, glutamate induced excitotoxicity during seizures, ischemia, stroke, cerebral hemorrhage, epilepsy, brain injury (head injury), hypoxia, and the like.

Therefore, in another embodiment, the present invention relates to a method of inhibiting glutamate receptors, preferably NMDA receptors, by inhibiting astrocytic G-protein coupled receptors, preferably PARs, more preferably PAR1, thereby inhibiting glutamate release from astrocytes, resulting in inhibiting glutamate receptors, preferably NMDA receptors, on adjacent postsynaptic neurons. In still another embodiment, the present invention relates to method for protecting nerve cells from glutamate neurotoxicity, by inhibiting astrocytic G-protein coupled receptors, preferably PARs, more preferably PAR1, and thereby inhibiting glutamate release from astrocytes. In still another embodiment, the present invention relates to method of preventing and/or treating and/or improving a disease caused by glutamate over-release induced neurotoxicity, wherein the disease is one or more selected from the group consisting of epileptic seizures, glutamate induced excitotoxicity during seizures, ischemia, stroke, cerebral hemorrhage, epilepsy, brain injury (head injury), hypoxia, and the like, by inhibiting astrocytic G-protein coupled receptors, preferably PARs, more preferably PAR1, inhibiting glutamate release from astrocytes, and inhibiting glutamate receptors, preferably NMDA receptors, on adjacent postsynaptic neurons.

With respect to this aspect, an embodiment of the present invention relates to a neuroprotecting agent for protecting nerve cells from glutamate neurotoxicity, wherein the agent contains as an active ingredient one or more antagonists and/or inhibitors against astrocytic G-protein coupled receptors, preferably PARs, more preferably PAR1, and has the effect of inhibiting over-release of glutamate from astrocytes and over-activation of glutamate receptors on adjacent postsynaptic neurons. In still another embodiment, the present invention relates to a composition for preventing and/or treating and/or improving one or more glutamate over-release associated diseases selected from the group consisting of epileptic seizures, glutamate induced excitotoxicity during seizures, ischemia, stroke, cerebral hemorrhage, epilepsy, brain injury (head injury), hypoxia, and the like, containing as an active ingredient one or more antagonists and/or inhibitors against astrocytic G-protein coupled receptors, preferably PARs, more preferably PAR1. The PAR1 antagonist or inhibitor may be one or more selected from the group consisting of BMS-200261 (trans-Cinnamoyl-F(f)-F(Gn)L-Arg-Arg-NH2, J Med Chem 1996 Dec. 6; 39(25):4879-87), peptide PPACK, hirudin, and the like. The effective amount of the PAR1 antagonist or inhibitor may be appropriately adjusted, and is preferably from 10 nM to 100 uM.

In another embodiment, the present invention relates to a method of screening a neuroprotecting agent, including the steps of:

selectively contacting a candidate compound with a G-protein coupled receptor on astrocyte;

measuring inhibition of a glutamate receptor, preferably an N-methyl-D-aspartic acid (NMDA) receptor, on an adjacent postsynaptic neuron; and

determining the candidate compound as a neuroprotecting agent for protecting nerve cells from neurotoxicity caused by over-release of glutamate from an astrocyte and over-activation of a glutamate receptor on adjacent postsynaptic neuron, when the glutamate receptor in the case of contacting the candidate compound is more inhibited compared with the case of not contacting the candidate compound. The screened neuroprotecting agent may be useful as an agent for preventing and/or treating one or more acute or degenerative brain diseases caused by glutamate over-release selected from the group consisting of epileptic seizures, glutamate induced excitotoxicity during seizures, ischemia, stroke, cerebral hemorrhage, epilepsy, brain injury (head injury), hypoxia, and the like.

The inhibition of glutamate receptors on adjacent postsynaptic neurons may be determined by an inward current change through the glutamate receptors on adjacent postsynaptic neurons before and after treating the candidate compound, where the inhibition is confirmed when the inward current change after treating the candidate compound is decreased. The inward current change for confirming the inhibition of glutamate receptors may be determined by all conventional manners known to the relevant art, for example a patch clamp and the like. In addition, the step of selectively contacting a candidate compound with G-protein coupled receptors on astrocytes may be performed by all conventional manners known to the relevant art. For example, the step may be done by treating the candidate compound after treating antagonists against all receptors on astrocytes other than the specific G-protein coupled receptor to be examined, thereby inactivating all the astrocytic receptors except the would-be examined receptor, but is not limited thereto.

The present invention is further explained in more detail with reference to the following examples. These examples, however, should not be interpreted as limiting the scope of the present invention in any manner.

### EXAMPLE 1

### Tissue Culture

1.1: Preparation of Wild-Type and PAR1−/− Astrocytes

Cultured astrocytes were prepared from P0-P3 postnatal mice obtained from KIST SPF. The cerebral cortex was dissected free of adherent meninges, minced, and dissociated into single cell suspension by trituration through a Pasteur pipette. All procedures involving the use of animals were reviewed and approved by the Emory University IACUC. Dissociated cells were plated onto either 12 mm glass coverslips or 6 well plates coated with 0.1 mg/ml poly-D-lysine. Cells were grown in a DMEM media (Gibco, cat# 11960-044) supplemented with 25 mM glucose, 10% heat-inactivated horse serum, and 10% heat-inactivated fetal bovine serum, 2 mM glutamine, and 1000 units/ml penicillin-streptomycin. Cultures were maintained at 37° C. in a humidified 5% CO₂-containing atmosphere. Astrocyte cultures prepared in this way were previously determined by GFAP (glial fibrillary acidic protein) staining to be greater than 95% astrocytes (Nicole et al., 2005). In some experiments, the culture media was replaced 24 hours after plating with DMEM with all added components except glutamine, and cultures were maintained for 4 days before experimentation in glutamine-free media.

1.2: Astrocyte-Neuron Co-Culture

For an astrocyte-neuron co-culture, a monolayer of wild-type astrocytes was grown to 70-100% confluency (7-14 days in culture). Subsequently, the cortex from a P0 to P5 PAR1−/− mouse (Connolly et al., 1996) was dissected free of meninges and digested for 30 min in 1 mg/ml trypsin at 37° C. PAR1−/− mice were >99% C57B1/6 (>7 backcrossings), and wild-type mice were from a colony derived from PAR1−/− littermates.

All experiments were done within 3 generations of establishing the homozygous colony. The cortical cells were plated at low density on top of the monolayer of wild-type astrocytes obtained in above Example 1.1. Neurons were used for Ca²⁺ imaging after 1-5 days in culture.

1.3: Preparation of GluR1(L497Y)-Transfected HEK 293 Cells

HEK 293 cells (ATCC1573) were plated onto 12 mm glass coverslips coated with 5-10 ug/ml poly-D-lysine and grown in a DMEM media (Gibco, cat# 11960-044) supplemented with 25 mM glucose, 10% heat-inactivated horse serum, and 10% heat-inactivated fetal bovine serum, 2 mM glutamine, and 1000 units/ml penicillin-streptomycin (Banke & Traynelis, 2000; Traynelis & Wahl, 1996). The obtained cultures were maintained at 37° C. in a humidified 5% CO₂-containing atmosphere.

HEK 293 cells were transfected with a 1:3.5 ratio of GFP and GluR1(L497Y) using the calcium phosphate method using effectence for 6-8 hours, after which the media was replaced and supplemented with 1 mM kynurenic acid and 10 μM N-(4-hydroxyphenylpropanoyl) spermine or 10 μM CNQX. The transfected HEK cells were subsequently trypsinized and replated onto astrocyte feeder layers derived from either wild-type or PAR1−/− mice 24 hours post-transfection, and recordings performed 24 hours after replating.

### Example 2

### Acute Dissociation of CA1 GFAP-GFP Astrocytes from Hippocampal Slices

The CA1 region was micro-dissected from hippocampal slices (300 μm thick, see below) and exposed for 30 min at 37° C. to 1 mg/ml trypsin (type III; Sigma, St. Louis, Mo.) dissolved in divalent free HEPES buffered saline. Trypsin was subsequently inactivated by adding an external solution containing CaCl₂ (2 mM) and MgCl₂ (2 mM). The hippocampal sections were mechanically dissociated with fire-polished glass pipettes. Cells were washed twice and plated on poly-D-lysine (10 ug/ml) coated glass coverslips, and placed in an incubator at 37° C. for 30 to 60 min before use.

### Example 3

### Perforated Patch

Whole-cell perforated-patch recording from cultured cortical neurons or HEK cells under voltage clamp (holding potential −60 mV) was made with an Axopatch 200B amplifier (Axon Instruments, Union City, Calif.). The recording chamber was continually perfused with a recording solution comprised of 150 mM NaCl, 3 mM KCl, 2 mM CaCl₂, 5.5 mM glucose, and 10 mM HEPES (pH 7.4 by NaOH; osmolality adjusted to 315-320 mOsm with sucrose). Recording electrodes (4-7 MΩ) were filled with (in mM) 150 mM CsMeSO₄, 10 mM NaCl, 0.5 CaCl₂, 10 mM HEPES, and 25-50 μg/ml gramicidin D (pH adjusted to 7.3 with CsOH and osmolality adjusted to 310 mOsm with sucrose). It took 20-30 min to achieve acceptable perforation with series resistance ranging from 30-60 MΩ. All electrophysiological data from cultured neurons cells in this study were collected at room temperature (23-26° C.).

### Example 4

### Electrophysiological Recording from Rat Hippocampal Slices

Young rats (Sprague-Dawley, age P15-P20) or mice (C57/B16, age P14-19) were deeply anesthetized with isoflurane and decapitated. The brain was rapidly removed and submerged in an ice-cold oxygenated artificial cerebrospinal fluid (ACSF) comprised of 130 mM NaCl, 24 mM NaHCO₃, 3.5 mM KCl, 1.25 mM NaH₂PO₄, 1 mM CaCl₂,3 mM MgCl₂, and 10 mM glucose saturated with 95% O₂/5% CO₂, at pH 7.4.

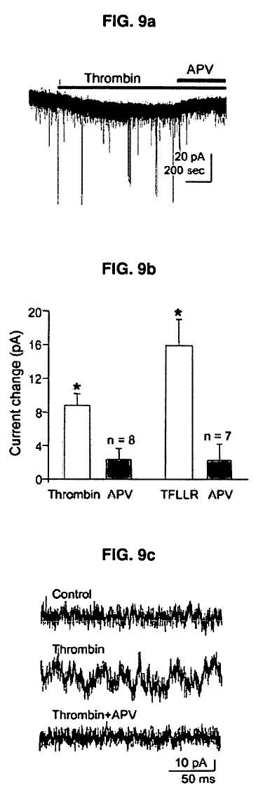

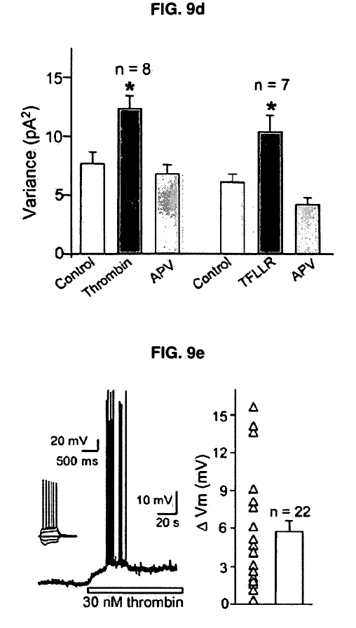

The hemisected brain was glued onto the stage of a vibrating microtome (Leica VT1000 S) and sections of 300 μm thickness were cut and stored in an incubation chamber at room temperature for about 1 h before use. For voltage clamp experiments, the solution used to fill the electrodes was comprised of 140 mM Cs-MeSO₄, 10 mM HEPES, 7 mM NaCl, 4 mM Mg-ATP, and 0.3 mM Na₃-GTP; the solution for current clamp recordings was comprised of 140 mM K-MeSO₄, 10 mM HEPES, 7 mM NaCl, 4 mM Mg-ATP, and 0.3 mM Na₃-GTP, and supplemented with 1 mM QX314; and 1 mM QX314 was omitted from some current clamp recordings (e.g., FIG. 9e).

Slices were placed on the stage of an upright microscope underneath a platinum and nylon restraining grid, and superfused with oxygenated ACSF at room temperature (23° C.), some experiments are performed at 34° C. by a temperature controller (TC-344B, Warner, Hamden). The standard ACSF recording solution was comprised of 130 mM NaCl, 24 mM NaHCO₃, 3.5 mM KCl, 1.25 mM NaH₂PO₄, 1.5 mM CaCl₂, 1.5 mM MgCl₂, and 10 mM glucose saturated with 95% O₂/5% CO₂, at pH 7.4. For some experiments 0.5-1 μM TTX, 10 μM NBQX, or 10-20 μM bicuculline were added to the extracellular solution, extracellular Mg²⁺ was reduced to 5 μM, and/or extracellular Ca²⁺ raised to 2 mM.

Visually guided whole-cell patch recordings were obtained from CA1 pyramidal neurons in a voltage clamp or current clamp configuration using an Axopatch 200A (Axon Instruments) and a borosilicate patch pipette of 5-7 MΩ resistance. All neurons included in this study had a resting membrane potential below −55 mV, an access resistance in the range of 10-20 MΩ, and showed only minimal variation in these parameters (resting membrane potential and access resistance) during the recording period. Records were filtered at 5 kHz and digitized at 20 kHz using a Digidata 1200 A/D board.

Synaptic responses were evoked by applying 0.1 ms current injection (1-100 μA) to a bipolar stimulating electrode placed in the stratum radiatum. Thrombin (Calbiochem) concentration was determined as described by Gingrich et al. (2000) using conversion of 1 U/ml=10 nM. TFLLR and thrombin were applied by gravity perfusion at 1.0 ml/min, with extensive washing of perfusion lines, chamber, and objective between experiments to remove all residual thrombin, which has been reported to irreversibly cleave and PAR1 at a pM concentration. Removal of all thrombin ensured that PAR1 receptors were not pre-cleaved by residual thrombin prior to experimentation.

### Example 5

### Miniature EPSC Analysis

Miniature EPSCs (mEPSCs) were digitized at 10 KHz and the records were filtered with a digital Gaussian filter (−3 dB) with a cutoff frequency of 1 kHz. The mEPSCs were automatically detected and grouped based on an amplitude threshold of 10 pA and a rise time between 0 to 5 (fast rise time) and 5 to 10 ms (slow rise time); a small number of apparent mEPSCs with rise times slower than 10 ms were not studied further. Selected mEPSCs were scaled, averaged, and fitted with a sum of two exponential functions,

Response Amplitude(t)=Amplitude₁exp(−time/τ₁)+Amplitude₂exp(−time/τ₂).

If the first decay constant (τ₁) and the second decay constant (τ₂) were within 10%, the curve was subsequently refitted with a single exponential function. Both the frequency and the peak amplitude of detected events were analyzed. The AMPA receptor blocker CNQX (10 μM) was routinely added at the end of experiments to verify that the mEPSCs were AMPA receptor-mediated. All data were acquired, stored, and analyzed using pClamp 8 (Axon Instruments, Foster City, Calif.) and Mini Analysis Program (Synaptosoft). In all of the experiments, drugs were administered by addition to the perfusing medium and were applied for a sufficient period to allow equilibration.

### Example 6

### Imaging of Voltage- and Ca²⁺-Sensitive Dyes

Cultured astrocytes were incubated with 5 M Fura2-AM in 1 M pluronic acid (Molecular Probes) for 30 min at room temperature, and subsequently transferred to a microscope stage for imaging. The external solution contained 150 mM NaCl, 10 mM HEPES, 3 mM KCl, 2 mM CaCl₂, 2 mM MgCl₂, and 5.5 mM glucose, and the pH was adjusted to pH 7.3 and osmolarity to 325 mOsm. Intensity images of a 510 nm wavelength were taken at 340 nm and 380 nm excitation wavelengths using either a Micromax Camera (Princeton) or an intensified video camera (PTI), and the two resulting images were used for ratio calculations using Axon Imaging Workbench version 2.2.1.

In order to evaluate Ca²⁺ signaling in neurons and glia in slices, patch electrodes were filled with 140 mM K-gluconate, 10 mM HEPES, 7 mM NaCl, 4 mM Mg-ATP, 0.3 mM Na₃-GTP, and either 100 μM Fluo-3 or Oregon Green 488 BAPTA-2 (Kd 580 nM). The external solution contained 1 μM TTX. Throughout the experiments, −5 mV voltage steps (15 ms duration) were applied at 30 sec intervals from a holding potential of −70 mV to continuously monitor the holding current, series resistance, and membrane input resistance. After entering whole-cell mode, the CA1 pyramidal cells in slices were maintained for 20 min to allow for dye filling before image acquisition using a Princeton Micromax camera.

After the baseline period, TFLLR (30-100 μM) was applied, and images were acquired every 3 sec with a 25 ms exposure to 450-490 nm light for each image. Ca²⁺-dependent fluorescence intensity (520 nm) was measured in cell bodies and processes by using Axon Imaging Workbench (v2.2.1) and expressed as ratio image of (F-Fo)/Fo, where Fo is the fluorescence intensity before drug treatment. Increases in fluorescence ratio of greater than 0.2 were considered to be significant changes; baseline fluorescence values possessed a peak (F-Fo)/Fo ratio on average of 0.01±0.01.

### Example 7

### Glutamate Release Assay

Astrocytes obtained in the above Example 1.1 were loaded with 0.5 μM L-3H-glutamate for 60 min by adding 1 μM of 1 mCi/ml L-3H-glutamate stock solution to 2 ml of DMEM culture media. The cultures were preincubated for 30 min with 1 mM amino-oxyacetic acid and 0.5 mM methionine sulfoximine before adding ³H-glutamate and during the loading to inhibit the metabolism of glutamate to glutamine and other metabolites (Farinelli and Nicklas, 1992).

Cells were washed with an external solution 3 times. In some experiments, the external solution was supplemented with 50 μM L-transpyrrolidine-2,4-dicarboxylic acid (trans-PDC) to block glutamate transporters, a maximally effective concentration (6×IC₅₀ of 4-8 μM; Mitrovic & Johnston, 1994;) that is well below that suggested to stimulate heteroexchange (0.2 mM; Volterra et al., 1996; Bezzi et al., 1998).

Agonists (TFLLR 30 μM) were added to the external solution for 6 min and the experiment was terminated by collection of the solution. Each experimental run included a control condition in which no agonist was added. Six replicates were obtained for each drug condition. For analysis, the average radioactivity count was obtained from 6 replicates for each condition and this average was compared to the average of the control condition.

### Experimental Example 1

### Measuring the Change of Intracellular Ca²⁺ Depending on Activation of PAR1 in Cultured Astrocytes

In this example, the experiment showing the increases in astrocytic intracellular Ca²⁺ evoked by activation of protease activated receptor-1 (PAR1), bradykinin receptors, and P2Y receptors, which are representative G-protein coupled receptors, was performed by bath perfusion of agonists. The obtained results show that activation of PAR1 in astrocytes causes the increase in the level of intracellular Ca²⁺ in astrocytes, which will be more specifically described as follows.

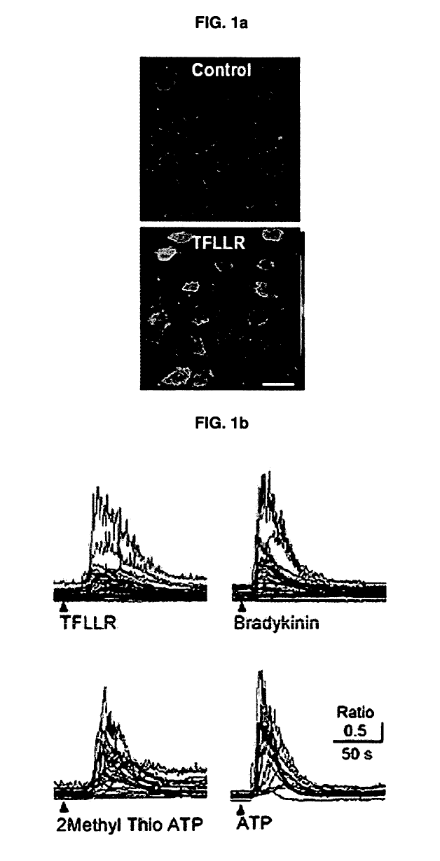

A Ca²⁺ sensitive dye Fura2-AM was loaded on the cultured wide-type mouse astrocytes obtained in the above Example 1.1 (see Example 6), subsequently 30 μM TFLLR, 10 μM bradykinin (Sigma), 50 μM 2-methyl-thio-ATP, and 10 μM ATP were added to the Fura2-AM-loaded culture, and then the fluorescence and image were observed. The results are shown in FIGS. 1a and 1b.

FIG. 1a shows a superimposed ratio image (510 nm emission; 340 nm/380 nm excitation) of Fura2-AM loaded cultured wild-type mouse astrocytes before and 20 s after 30 μM TFLLR application. The color scale shows the pseudocolor coding of ratio values ranging from 0 (bottom) to 3 (top). The calibration bar is 50 μm. FIG. 1b shows representative traces of ratio amplitude changes in the Fura-2 fluorescence ratio by pressure application of a brief pulse of 30 μM TFLLR, 10 μM bradykinin, 50 μM 2-methyl-thio-ATP, and 10 μM ATP. As shown in FIGS. 1a and 1b, the applications of TFLLR, bradykinin, 2-methyl-thio-ATP, and ATP all increase the level of astrocytic intracellular Ca²⁺. Since the materials have been known as G-protein coupled receptor activators, the result shows that the activation of G-protein coupled receptor (such as PAR1, etc.) causes an increase in the level of astrocytic intracellular Ca²⁺.

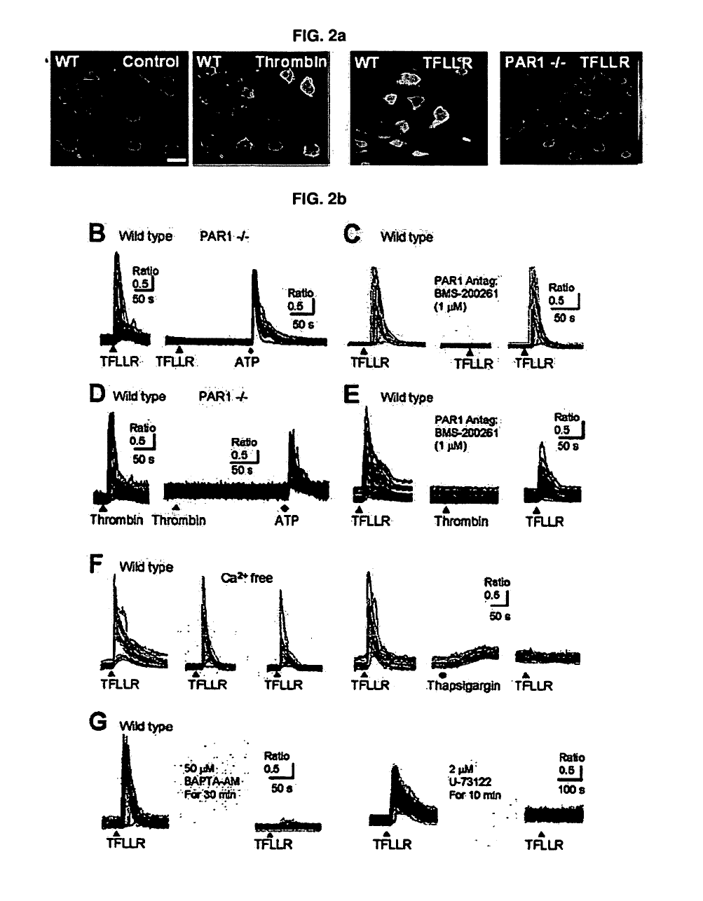

In addition, the results obtained by observing the intracellular Ca²⁺ signaling in response to PAR1 activation in the cultured astrocytes are shown in FIG. 2. In FIG. 2a, the left two panels show a ratio image of control and Fura2-AM loaded cultured wild-type mouse astrocytes before and 20 s after 30 nM thrombin application; and the right two panels show ratio images 20 s after 30 μM TFLLR on wild-type and PAR1−/− mouse astrocytes. Ratio calibration is 0-3; the calibration bar is 50 μm. As shown in FIG. 2a, it was found that all wild-type mouse astrocytes showed a robust increase in fluorescence of the Ca²⁺ sensitive dye Fura-2 in response to the application of 30 nM thrombin and 30 μM of the selective PAR1-activating peptide TFLLR (at least 3-fold EC₅₀; Hollenberg et al., 1997).

FIG. 2b shows superimposed representative ratio response time courses that show magnitude of the changes in Fura2 fluorescence ratio of wild-type and PAR1−/− astrocyts, when 30 nM thrombin (indicated as a filled triangle), 30 μM TFLLR (indicated as a filled triangle), and/or 10 μM ATP (indicated as a filled lozenge) are added. In FIG. 2b, B shows superimposed representative ratio response time courses that show the magnitude of the changes in Fura2 fluorescence ratio of wild-type and PAR1−/− astrocyts, wherein 30 μM TFLLR was added for 10 s and TFLLR was applied at 10 min intervals to minimize desensitization. TFLLR application did not cause an increase in intracellular Ca²⁺ concentration in PAR1−/− astrocytes, although astrocytes still responded to the positive control ATP. C is the result from the pretreatment of 1 μM of a PAR1 antagonist, BMS200261, on wild-type astrocytes before the treatment of TFLLR, showing that BMS 200261 reversibly and completely antagonized the TFLLR effects on wild-type astrocytes. D shows representative traces of Fura2 ratio of wild-type and PAR1−/− astrocytes by 30 nM thrombin, wherein thrombin application did not cause an increase in intracellular Ca²⁺ concentration in PAR1−/− astrocytes, although astrocytes still responded to the positive control 10 μM ATP. E shows the results from the pretreatment of 1 μM BMS200261 on wild-type astrocytes before the treatment of thrombin, indicating that 30 nM thrombin has no significant effect on wild-type astrocytes pre-treated with 1 μM of the PAR1 antagonist BMS 200261, and the change in Fura-2 ratio caused by BMS+thrombin was 16.7±3.3%, n=116 cells from 4 coverslips, compared to a change by thrombin of 150.9±17.4%, n=76 cells from 3 coverslips, p<0.01. TFLLR applied before and after application of thrombin served as a positive control. F shows the change in Ca²⁺ concentrations depending on the application of TFLLR in the nominal absence of extracellular Ca²⁺, indicating the application of TFLLR (30 μM) increased intracellular Ca²⁺ concentration, and pre-exposure to 1 μM thapsigargin blocked the subsequent response to TFLLR application. G shows the change in Ca²⁺ concentrations in the case that mouse astrocytes treated with 50 μM BAPTA-AM (Molecular Probes) for 30 min after an initial TFLLR application, indicating that such BAPTA-AM treatment blocked the Ca²⁺ response to subsequent TFLLR application, and the treatment of the PLC inhibitor U-73122 (2 μM) for 10 min similarly blocked the effects of TFLLR.

FIG. 2 may be summarized as follows. That is, both of the agonist peptide of which the agonist peptide TFLLR (30 uM) and the serine protease thrombin (30 nM) selectively activate PAR1, having no effect in PAR1−/− mice (n=3 for TFLLR, n=5 for thrombin; B and D). ATP served as a positive control in PAR1−/− mice. In addition, 1 μM of the antagonist BMS200261 (Bernatowicz et al., 1996; Kawabata et al., 1999) can selectively and reversibly block PAR1 activation by either 30 μM TFLLR (C; n=4 experiments; p<0.01) or 30 nM thrombin (E; n=4 experiments; p<0.01). These data suggest that a modest concentration of thrombin (30 nM) was relatively selective for PAR1 activation, since removal or blocking of PAR1 eliminated the intracellular Ca²⁺ response in cultured astrocytes. Further, the TFLLR-induced Ca²⁺ responses persisted in the absence of external Ca²⁺ (F, n=4) and were blocked by pretreatment with 1 μM thapsigargin (F; n=4). Treatment with the cell permeable Ca²⁺ chelator, BAPTA-AM (1,2-bis(2-aminophenoxy)ethane-N,N,N′,N′-tetra-acetic acid acetoxymethyl ester, 50 μM), eliminated the PAR1-evoked fluorescent response of Ca²⁺ sensitive dyes (G; n=4). Finally, 2 μM of the phospholipase C inhibitor U73122 (G; n=3) blocked TFLLR-induced increases in Ca²⁺ responses, suggesting that PAR1 stimulates Ca²⁺ release from inositol triphosphate-sensitive stores.

### Experimental Example 2

### Measurement of Intracellular Ca²⁺ Depending on Activation of Glial PAR1 in Brain Slices

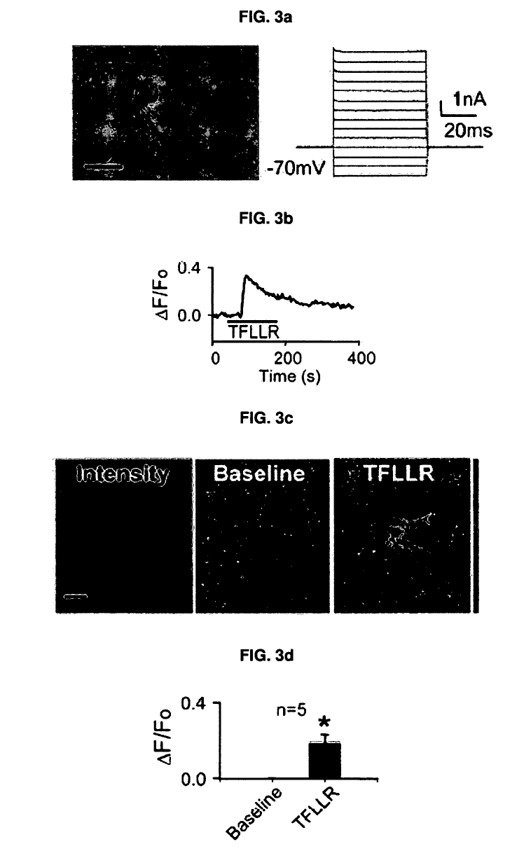

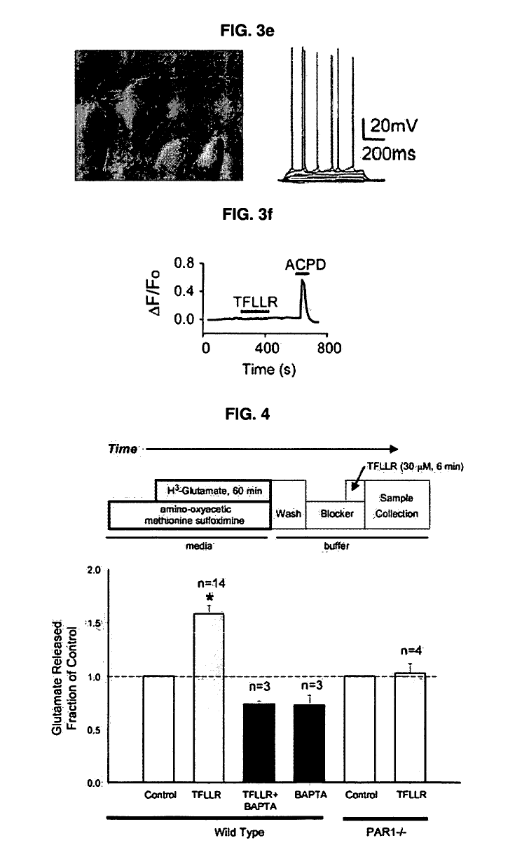

To test the hypothesis that PAR1 signaling pathways similar to those in the cultured astrocytes (in vitro) as described in Experimental Example 1 occur in brain tissue, the effects of PAR1 activation in neurons and astrocytes in acutely prepared rat brain slices were measured. As results, it was confirmed that intracellular Ca²⁺ concentration increases by the activation of PAR1 in the astrocytes in brain slices as well as the cultured astrocytes (see FIGS. 3a to 3f).

FIGS. 3a to 3f show that TFLLR increases intracellular Ca²⁺ concentration in glial cells in brain tissue, but not in CA1 pyramidal cells in hippocampal slices.

In FIG. 3a, the left panel shows a DIC image of a glial cell patched with 100 μM Oregon Green 488 BAPTA-2 included in the patch pipette (16 MΩ input resistance; V_{H}=−70 mV; V_{M}=−76 mV); and the right panel shows the results obtained by applying 10 mV voltage steps to this cell under voltage clamp, suggesting a linear current voltage relationship with no activation of time-dependent currents. The calibration bar is 10

FIG. 3b shows the changes in fluorescent intensity in the glial cell in (3a) during application of 30 μM TFLLR.

FIG. 3c shows fluorescent intensity in a glial cell (left, Intensity), and fluorescent change as a ΔF/Fo (as in 3b) before (center, Baseline) and after 30 μM TFLLR application (right, TFLLR). The baseline image is generated by taking the ratio of (F-Fo)/Fo, where Fo is the first intensity image. During TFLLR application the ratio increased significantly in the soma as well as the processes. The color scale on the right shows the pseudocolor coding of ratio values ranging from 0 (bottom) to 0.4 (top). The arrow in the left panel of 3c shows where the changes in fluorescence are monitored.

FIG. 3d shows the average changes in fluorescence (±SEM). * p<0.05, paired t-test.

FIG. 3e shows a DIC image of a CA1 pyramidal neuron (left panel), patched with 100 μM Fluo-3 included in the patch pipette (V_{M}=−62 mV, left panel). The right panel shows that 10 pA current injection steps were performed under applied current clamp and the membrane voltage changes and action potentials in this neuron.

FIG. 3f shows the changes in somatic fluorescent intensity in dye-loaded CA1 neurons during application of 30 μM TFLLR and 100 μM trans-ACPD (1-2-amino-4-phosphonbutanoic acid) (Tocris), wherein a broad spectrum metabotropic glutamate receptor agonist was used as a positive control.

Glial cells were identified by their small somatic size and distinct morphology (FIG. 3a left panel), negative resting potential, lack of voltage-dependent currents, and low input resistance (FIG. 3a right panel). In addition, CA1 pyramidal cells were identified by their location in stratum pyramidale, their morphology (FIG. 2e left panel), and the presence of action potentials upon a series of injected current steps (FIG. 3e right panel).

In this experimental example, no significant increase in somatic fluorescence of 2+Ca²⁺-sensitive dyes to application of thrombin (30 nM) or TFLLR (30 μM) to CA1 pyramidal neurons in hippocampal slices (FIGS. 3e and 3f; n=17) was detected. Either the broad spectrum metabotropic glutamate receptor (mGluR) agonist trans-ACPD (100 μM) or the group I mGluR agonist (S)-3,5-dihydroxyphenylglycine (30 μM) served as a positive control; it was found that both agonists evoked strong increases in somatic neuronal Ca²⁺ fluorescence in all cells tested.

Imaging of the Ca²⁺ sensitive dye Fura2-AM loaded into acutely dissociated cells from the CA1 region dissected from hippocampal slices was additionally performed, to further screen for TFLLR responsive cells (see Example 6). It was found that twenty-four (24) of twenty-five (25) TFLLR-responsive cells were unresponsive to NMDA, suggesting they were non-neuronal. This is in striking contrast to only one (1) of twenty-four (24) NMDA-responsive neurons that showed a response to PAR1 activation. These results together suggest that functional coupling of PAR1 to Gαq/11-mediated Ca²⁺ signaling is largely restricted to glial cells in the CA1 region of the hippocampus.

### Experimental Example 3

### Effect of Activation of Astrocytic PAR1 on Ca²⁺-Dependent Release of Glutamate

Based on the fact that the activation of astrocytic PAR1 increases the intracellular Ca²⁺ concentration as shown in the above Experimental Example 1, it was tested in this example whether the activation of astrocytic PAR1 stimulates the release of glutamate in cultured astrocytes. As results, it was found that the activation of astrocytic PAR1 stimulates Ca²⁺-dependent release of glutamate in astrocytes, which is shown in FIGS. 4-7.

3.1: Quantitative Analysis of Glutamate Release

FIG. 4 shows that PAR1 activation stimulates Ca²⁺-dependent release of glutamate in astrocytes. The top panel schematically shows the experimental protocol for assaying glutamate release from cultured astrocytes. To inhibit the conversion of glutamate to glutamine and other metabolites, amino-oxyacetic acid (1 mM) and methionine sulfoximine (0.5 mM) were pre-incubated for 30 min and included throughout the loading of ³H-glutamate. Cells were washed with an external solution and subsequently TFLLR (30 μM) was added for 6 min. In some experiments, the external solution was supplemented with 50 μM trans-PDC (50 μM, IC₅₀=4-8 μM; Mitrovic & Johnston, 1994; Esslinger et al., 2002) to block the glutamate transporter, a level well below that known to stimulate heteroexchange (Volterra et al., 1996; Bezzi et al., 1998). In the top panel, the thick boxes represent cells in culture media and the white boxes represent cells in external solution.

The bar graph at the bottom of FIG. 4 shows that TFLLR induced a significant increase in glutamate release that was blocked by 30 min treatment of 50 μM BAPTA-AM. The increase in glutamate release by TFLLR was absent in PAR1−/− astrocytes, indicating that the increase in glutamate release by TFLLR is caused by the activation of PAR1. Numbers on top of each bar indicate the number of 6-well plates. * p<0.05, ANOVA for wild-type, unpaired t-test for PAR1−/−.

As shown in FIG. 4, release of glutamate, as measured by the efflux of radiotracer from [³H]-glutamate loaded cultures (Duan et al., 2003), was significantly increased upon TFLLR application by 56±9% compared to the control untreated with TFLLR (n=14). This increase in glutamate release was not observed in astrocyte cultures prepared from PAR1−/− mice (n=4), and was blocked by BAPTA-AM treatment (n=3), indicating the PAR1 selectivity and Ca²⁺-dependence of glutamate release. Glutamate release was measured in both the absence and presence of a maximally effective concentration of the glutamate uptake blocker trans-PDC (50 μM, IC₅₀=4-8 μM; Mitrovic & Johnston, 1994; Esslinger et al., 2002) and was indistinguishable, suggesting that release did not reflect reversal of the glutamate uptake pump. The level of trans-PDC utilized in this experimental example was below the threshold level proposed to stimulate heteroexchange (0.2 mM; Volterra et al., 1996; Bezzi et al., 1998).

3.2: Quantitative Measurement of Release of Glutamate by Sniffer-Patch Detection System

3.2.1: Measurement of Glutamate Release from Cultured Astrocytes

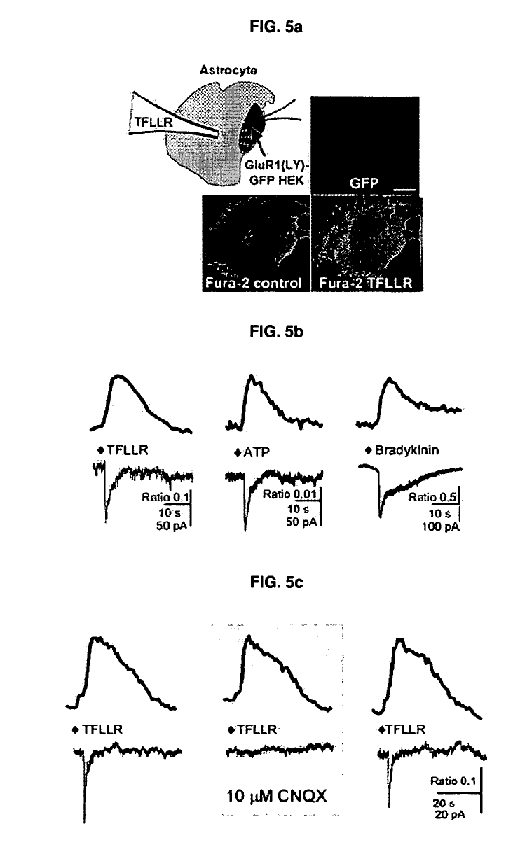

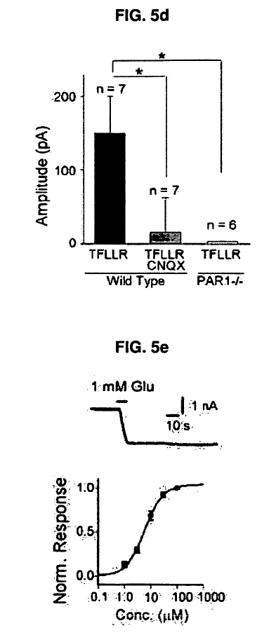

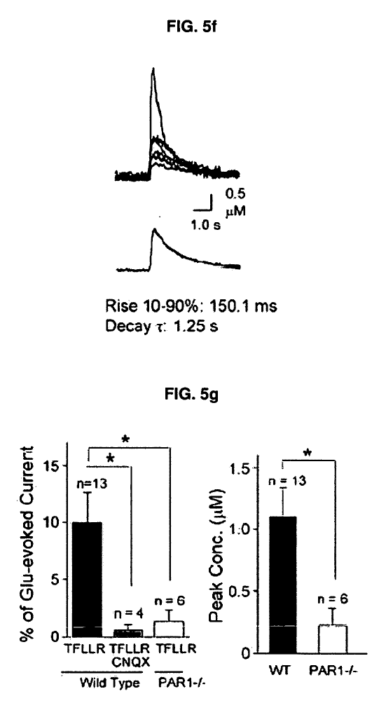

In this experimental example, the PAR1-stimulated glutamate release was quantitatively evaluated in real time by a “sniffer-patch” detection system. In the system, HEK 293 cells (see Example 1.3) transfected with the non-desensitizing GluR1 mutant L497Y (Stem-Bach et al., 1998) were used as a biosensor of glutamate release from cultured cortical astrocytes (in FIG. 5a). GluR1(L497Y) responds to glutamate with a sustained current that will temporally follow the glutamate concentration. Thus, this technique provides millisecond time resolution for the detection of micromolar levels of glutamate released from astrocytes.

GluR1(L497Y)-transfected HEK cells were directly plated onto an astrocyte monolayer, and subsequently the whole cell HEK current response under voltage clamp during a brief 0.2 sec application of the PAR1 activator TFLLR (500 μM), ATP (300 μM), or bradykinin (180 μM), respectively, from a pressurized pipette was recorded (FIGS. 5a, and 5b).

FIGS. 5a and 5b show the use of GluR1(L497Y) transfected HEK cells as biosensors for astrocytic glutamate release and the measured results thereby.

FIG. 5a is a schematic illustrating experimental setup and a GFP fluorescent image of astrocyte—GluR1(L497Y)/GFP transfected HEK cell co-culture (upper left panel). The lower panel shows the ratio image (510 nm emission; 340 nm/380 nm excitation) of Fura2-AM loaded co-cultures (Example 2.1) before and after brief (<1 sec) pressure-applied TFLLR (500 μM in pipette). The calibration bar is 20 μm.

FIG. 5b shows the results of the quantification of the fluorescence increase in response to brief (<1 sec) pressure application of 500 μM TFLLR, 300 μM ATP, and 180 μM bradykinin in wild-type astrocytes (upper trace) recorded together with the inward current induced in adjacent GluR1(L497Y)-transfected HEK cell (lower trace). Ca²⁺ sensitive dye Fura-2-AM recordings revealed that TFLLR, ATP, and bradykinin all increased astrocytic intracellular Ca²⁺ and elicited an inward current in HEK cells expressing GluR1(L497Y)

FIG. 5c shows Fura2 fluorescence ratio (upper trace) and inward current (lower trace) when 10 μM of the AMPA receptor antagonist CNQX (Tocris) was applied. As shown in FIG. 5c, CNQX (Tocris) reversibly inhibits TFLLR-evoked inward current in GluR1(L497Y) in a representative transfected HEK cell (n=7).

FIG. 5d shows the results of the response in GluR1(L497Y) transfected HEK cells to TFLLR application to astrocytes as peak current in wild-type and PAR1−/− astrocyte cultures. In wild-type astrocytes and co-cultured GluR1(L497Y)-transfected HEK cells, the current response is maximum when TFLLR is applied, and the application of 10 μM CNQX significantly reduced the response of GluR1(L497Y)-transfected HEK cells to TFLLR application (n=7; p<0.05, one way ANOVA). There is virtually no detectable current response recorded from GluR1(L497Y)-transfected HEK cells co-cultured with PAR1−/− astrocytes in response to pressure application of TFLLR (3.4±0.8 pA, n=6, p<0.05, one way ANOVA, compared with wild-type response amplitude).

FIG. 5e shows the dose response relationship and current response to pressure application of TFLLR converted to concentration using the dose response relationship and maximal current response of the GluR1(L497Y) transfected HEK cell as described in following Formula 1:

Concentration(t)=EC₅₀[response(t)/(100−response(t))]^{(1/n)} (Formula 1)

(where response(t) is the response amplitude expressed as a percent of the maximum achievable response and n is the Hill slope.)

As evaluated by the above formula, EC₅₀ value for glutamate activation of GluR1(L497Y) in transfected HEK cells was 6.1 μM (Hill slope 1.3).

FIG. 5f shows the concentration responses from 7 cells superimposed (upper panel) and below as an average (lower panel).

FIG. 5g summarizes the glutamate evoked current response (%) and the peak concentration in GluR1(L497Y) transfected HEK cells to TFLLR application to wild-type and PAR1−/− astrocytes. The glutamate evoked current (%) was 10±2.6% (n=13) when TFLLR was applied to wild-type; 0.64±0.47% when TFLLR+CNQX was applied to wild-type; and 1.4±0.9% when TFLLR was applied to PAR1−/−, * p<0.05, one-way ANOVA), and the maximum concentration was 1.1±0.24 μM and 0.23±0.13 μM in wild-type and PAR1−/− astrocyte cultures, respectively. * p<0.05, unpaired t-test.

As shown in FIGS. 5e and 5g, from simultaneous imaging of Ca²⁺-sensitive fluorescent dyes, it was confirmed that TFLLR did not alter PAR1−/− astrocytic intracellular Ca²⁺ but did increase HEK cell intracellular Ca²⁺, as expected given endogenous expression of PAR1 in HEK cells. These control experiments were performed on the same day as TFLLR stimulation of HEK cells on wild-type astrocytes, and confirm that PAR1 activators had no direct effect on GluR1(L497Y) currents in HEK cells.

Using the above Formula 1, it was estimated that glutamate reaches a peak value at an average of 1.1 μM, and decays with an approximately exponential time course (FIGS. 5f and 5g), which in this system is a complex reflection of diffusion, uptake, and intracellular astrocytic Ca²⁺ dynamics. Bradykinin and ATP induce glutamate concentrations approaching 3.9 μM and 5.9 μM, respectively. The results in FIG. 5g strongly suggest that activation of astrocytic PAR1 can stimulate glutamate release from astrocytes by a Ca²⁺-dependent mechanism, and that levels of extracellular glutamate are sufficient to activate glutamate receptors.

3.2.2: Effect of Glutamate in Culture Media of Astrocytes

Two experiments were performed using the above sniffer-patch detection system to verify that the astrocytic release of glutamate observed did not reflect a culture artifact.

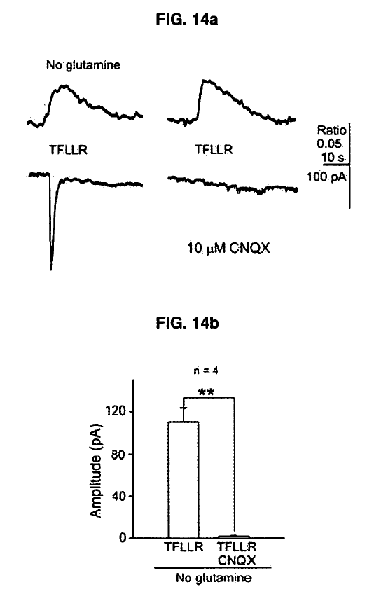

First, cultures were prepared in the absence of glutamine, which should prevent artifactual elevation of intracellular glutamate concentration that might have skewed levels of glutamate release observed. The TFLLR-induced glutamate release in glutamine-free culture media is shown in FIGS. 14a and 14b. FIG. 14a shows representative traces of TFLLR-induced fluorescence increase in wild-type astrocyte (upper trace) recorded together with the inward current from GluR1(L497Y) transfected HEK cell (lower trace), which are co-cultured in glutamine-free medium. FIG. 14b shows a summary of the amplitude changes by TFLLR and CNQX in a glutamine-free medium; ** p<0.01, paired t-test.

As shown in FIGS. 14a and 14b, TFLLR induced a current response that was 9.7±1.3% (n=4) of the maximal response from astrocytes cultured in the absence of glutamine, which was not significantly different than 10±2.6% (n=13; p>0.05) in the presence of glutamine. In addition, CNQX completely abolished the inward currents evoked by TFLLR, indicating that TFLLR application evoked identical CNQX-sensitive responses in GluR1(L497Y) transfected HEK cells when astrocytes were cultured in glutamine-free media. These results suggest that TFLLR-induced glutamate release was not solely the result of potentially high intracellular glutamate that might arise in a glutamine-supplemented culture media.

3.3: Glutamate Release from Astrocytes in Hippocampal Slices

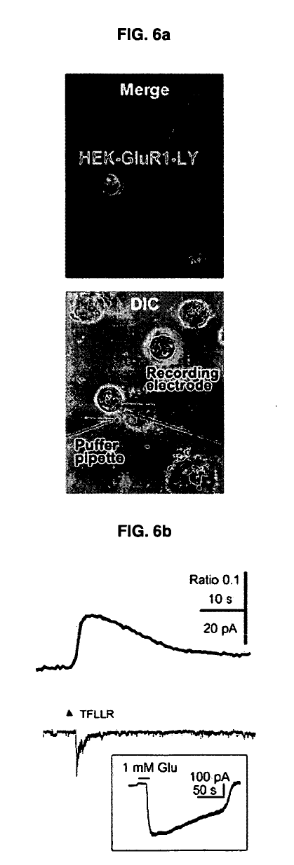

Cells were acutely dissociated from the CA1 region of hippocampal slices prepared from transgenic mice (Jackson Laboratories) expressing GFP under control of the GFAP promoter (Brenner et al., 1994), allowing unambiguous identification of isolated hippocampal astrocytes that had not been subject to tissue culture. Cells were dissociated directly onto GluR1(L497Y)-transfected HEK cells obtained in Example 1. Subsequently, GFP-expressing astrocytes that came to rest adjacent to a GluR1(L497Y)-transfected HEK cell (FIG. 6a) were identified, and patch clamp recordings from the GluR1(L497Y)-transfected HEK cell were used to detect glutamate release from the astrocytes.

TFLLR-evoked glutamate release from acutely dissociated CA1 astrocytes is shown in FIGS. 6a and 6b. In FIG. 6a, the upper panel shows the images of acutely dissociated GFAP-GFP labeled astrocytes (green) plated onto GluR1(L497Y) transfected HEK cells (red), and the lower panel shows a DIC image of the recording electrode and pressurized agonist filled pipette in the same co-culture as above. FIG. 6b shows representative traces of Fura-2 fluorescence increase in a GFAP-GFP labeled astrocyte (upper trace) recorded together with the inward current from GluR1(L497Y) transfected HEK cell (lower trace) in response to brief (1 sec) application of TFLLR. The inset shows the response to 10 s application of a maximally effective concentration of glutamate (1 mM) on the same cell.

FIGS. 6a and 6b show the results from a representative experiment in which brief application of the selective PAR1 activator TFLLR from a pressurized pipette evoked a response in nearby GluR1(L497Y)-transfected HEK cells. Similar results were found in 4 cells (1.46+0.54 μM; n=4), which confirms that glutamate release may occur from astrocytes in slices.

3.4: Comparison the Glutamate Releases in Neurons and Astrocytes

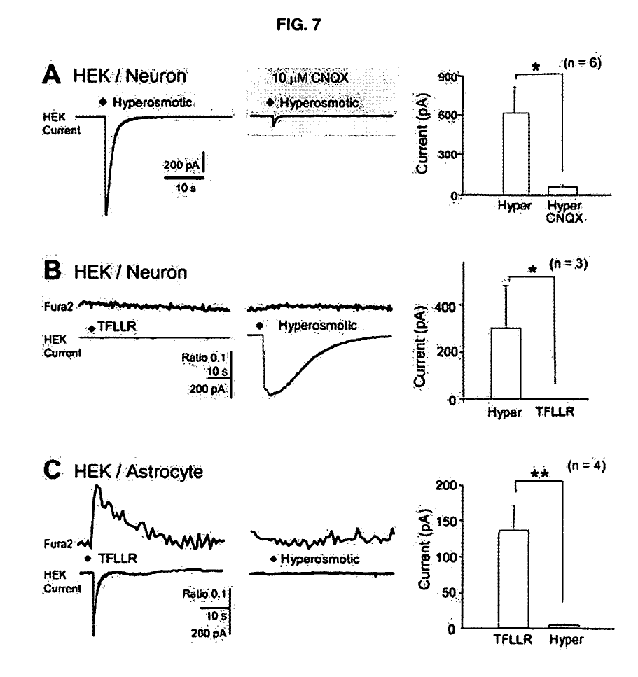

Because PAR1 activators induce little or no intracellular Ca²⁺ signaling in CA1 pyramidal cells or acutely dissociated CA1 neurons, it may be predicted that PAR1 activators will not induce glutamate release from neurons. To verify this prediction, effects of a hyperosmotic solution on the glutamate-release from cultured neurons and astrocytes were evaluated and are shown in FIGS. 7a to 7c.

FIG. 7a shows the ability of GluR1(L497Y)-transfected HEK co-culture system to detect CNQX-sensitive glutamate release from neurons in response to hyperosmotic solutions (530 mOsm, n=6), which stimulate vesicular release of glutamate from cultured central neurons. The left panel shows the detection results of neuronal glutamate release by using hyperosmotic solution (530 mosmol, H.O.) and GluR1(L497Y)-transfected HEK cell. The detection is abolished by the treatment of 10 μM CNQX. The right panel shows a summary of the current amplitude changes; * p<0.05; paired t-test (n=6). In all panels, inset is the response of a GluR1(L497Y)-transfected HEK cell to application of maximally effective concentration of glutamate (1 mM).

FIG. 7b shows that an HEK cell expressing GluR1(L497Y) may detect the neuronal glutamate upon the treatment of the hyperosmotic solution (301.6±179.3 pA, n=3), however it shows no response to application of 30 μM TFLLR (3.0±0.1 pA, n=3). * p<0.05; paired t-test. That is, a brief application of TFLLR caused no detectable glutamate-induced current in GluR1(L497Y)-transfected HEK cells adjacent to neurons.

FIG. 7c shows the results of the same day glutamate responses to activation of PAR1 in astrocytes, as a positive control for TFLLR activation of PAR1, wherein an HEK cell expressing GluR1(L497Y) may detect the astrocytic glutamate release from astrocytes during application of TFLLR (136±35 pA, n=4), however it shows virtually no response to application of the hyperosmotic solution (4.8±1.4 pA, n=4). ** p<0.01; unpaired t-test.

### Comparative Example

### Measurement of Glutamate Release Using NR1/NR2A Transfected HEK Cells

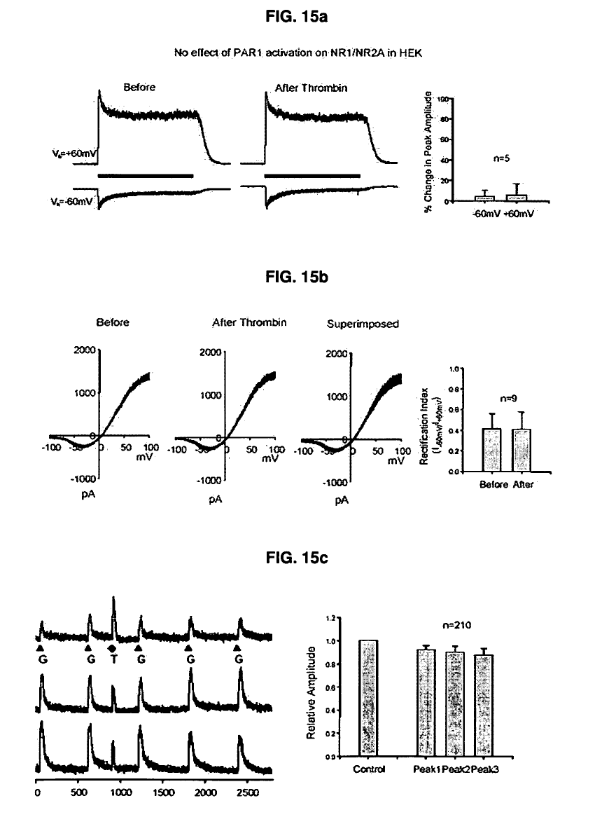

The effects of PAR1 activators on NMDA receptor responses in neurons as well as the effect of activation of endogenous PAR1 in HEK cells on recombinant NR1/NR2A receptors were examined. No change in neuronal or recombinant NMDA receptor response properties was detected before and after thrombin treatment. That is, PAR1 activation has no effects on NR1/NR2A transfected HEK cells. The obtained results are shown in FIGS. 15a to 15c.

FIG. 15a shows the record for NR1/NR2A transfected HEK 293 cells under gramicidin-D perforated patch, voltage clamp configuration. Current was induced by applying 5 mM glutamate while the cell was held at −60 mV and +60 mV. The external solution included 0.2 mM MgCl₂, 2 mM CaCl₂, 100 nM ZnCl₂, and 50 μM glycine. The horizontal bars indicate the duration of glutamate application. The response from the same cell is shown after a treatment of 30 nM thrombin for 30 s. The bar graph on the right shows pooled data from 5 cells. The peak amplitudes of glutamate-induced currents were compared before and after thrombin treatment at −60 mV and +60 mV holding potentials. As shown in FIG. 15a, there is no change in the current alteration profile in NR1/NR2A transfected HEK cell before and after treating the PAR1 activator thrombin.

FIG. 15b shows the I-V relationship obtained by applying voltage ramps from +100 mV to −100 mV and subtracting the traces before from during glutamate application on different HEK 293 cells. After 30 nM thrombin treatment the I-V relationship was similarly obtained and compared to the I-V relationship before the thrombin. I-V relationships before and after thrombin treatment were superimposed for comparison. The bar graph in the right panel shows the average of rectification index calculated by determining the ratio of current at −60 mV over at +60 mV and averaging across different cells. In this example, similar experiments using acutely dissociated or cultured hippocampal and cerebellar granule cells were attempted, but no modulation by PAR1 activation on NMDA receptor mediated currents was observed.

FIG. 15c is an image of Fura2 fluorescence on HEK cells expressing NR1/NR2A. Glutamate (G) was applied at 50 μM together with 50 μM glycine. Thrombin (T) was applied at 30 nM. The peak amplitude of fluorescence of the 2+Ca²⁺-sensitive dye was measured for each glutamate application and the changes in peak amplitude were calculated by taking the ratio of the glutamate response following the thrombin treatment over the average peak before the thrombin treatment.

These results suggest that PAR1 signaling in mammalian cells does not directly lead to posttranslational modification of the receptor, in contrast with previous conclusions from studies of PAR1 and NMDA receptors coexpressed in Xenopus laevis oocytes (Gingrich et al. 2000). Because it is possible to replicate potentiation in the oocyte but NMDA receptor potentiation in mammalian cells is not observed, it may be concluded that the intracellular signaling pathways linked to PAR1 in these two cells must differ.

### Experimental Example 4

### Effect of Astrocytic PAR1-Mediated Glutamate Release on Neuronal NMDA Receptors in Culture

To test whether astrocyte-released extracellular glutamate rises to sufficient levels to activate NMDA receptors on neuronal dendrites, the co-culture system was modified as described above, replacing GluR1(L497Y)-transfected HEK cells with cortical neurons derived from PAR1−/− animals growing on top of a wild-type astrocyte monolayer that was determined to be >95% GFAP positive cells (Nicole et al., 2005, Examples 1.1 and 1.2).

No NMDA responsive Fura-2 loaded cells were detected in the wild-type astrocyte monolayers before plating of neurons from PAR1−/− mice. These results show that all neurons in subsequent co-cultures were derived from PAR1−/− animals and did not arise as contaminating wild-type neurons from preparation of the astrocyte monolayer. Use of PAR1−/− neurons allowed evaluation of NMDA receptor responses to TFLLR-induced glutamate release without the confounding variable of potential intra-neuronal PAR1 activation.

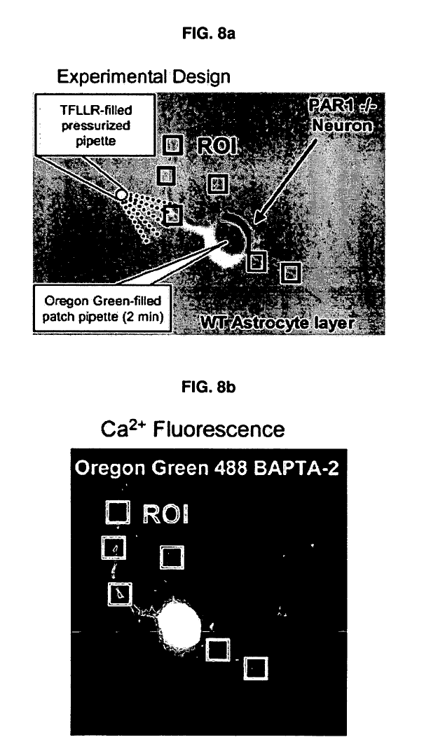

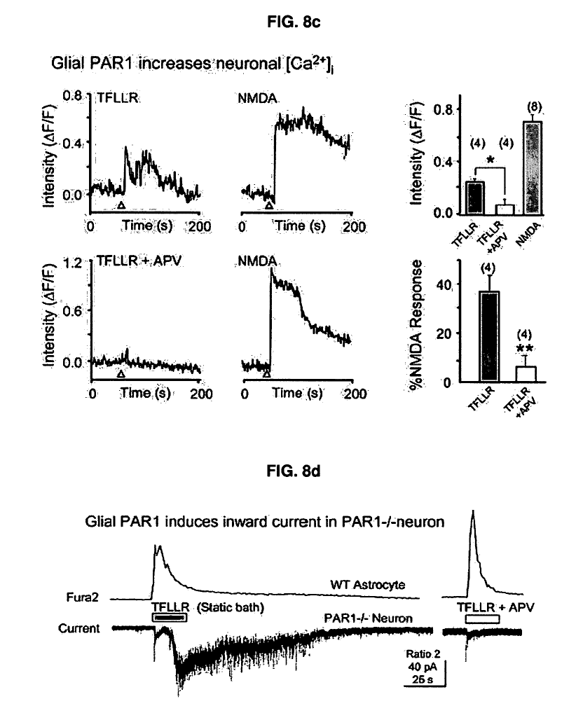

An individual neuron was dye-loaded through a patch electrode containing 100 μM of the Ca²⁺ indicator dye Oregon Green 488 BAPTA-2 (Kd=580 nM) for 1-2 min after breakthrough, and the patch electrode was subsequently withdrawn from the cell. After a 20 min recovery, the bath perfusion was stopped and TFLLR was applied by brief (<1 sec) pressure ejection onto the astrocyte hosting the dye-loaded neuron in a static bath condition. Changes in fluorescence were monitored in a number of dendritic processes (schematized in FIGS. 8a and 8b).

The results of monitoring are shown in FIGS. 8a and 8b. FIG. 8a shows a photomicrograph of a PAR1−/− cortical neuron that was loaded with 300 μM Oregon Green BAPTA2 for 2 min through a patch pipette after breaking the gigaohm seal. Several regions (boxes) at distal dendrites were imaged while 300 μM TFLLR was pressure-applied briefly (1 sec) from a pipette to surrounding wild-type astrocytes.

FIG. 8b shows a fluorescent image of the same PAR1−/− neuron, loaded with Oregon Green 488 BAPTA-2 (450-490 nm excitation; 520 nm emission). As shown in FIGS. 8a and 8b, TFLLR application increased the intra-neuronal Ca²⁺ concentration throughout the dendrites.

FIG. 8c shows the average fluorescent intensity response from 6 regions of interest on dendrites during brief TFLLR pressure application (marked by triangle; upper left traces). TFLLR applied in the presence of 100 μM DL-APV had no effect on intracellular Ca²⁺ in dendrites (lower left traces). 50 μM NMDA caused a saturating response in the same regions of interest. The peak fluorescent intensity change was obtained in response to TFLLR for each neuron, and mean values are compared for TFLLR, TFLLR in the presence of APV, and NMDA as a bar graph (upper right panel). There was a significantly larger Ca²⁺ increase with TFLLR than with TFLLR in the presence of APV, * p<0.01, paired t-test. Numbers in parentheses indicate the number of neurons tested. Data from the same experiment were also normalized to the response to NMDA application (lower right panel); ** p<0.05, paired t-test.

As summarized in FIG. 8c, TFLLR-induced increases in Oregon Green BAPTA-2 fluorescence may be observed in the dendrites, and this increase was blocked by the competitive NMDA receptor antagonist, D-2-amino-5-phosphono-valeric acid (APV; 50 μM).

Complimentary experiments were performed with the same culture system in which PAR1−/− neurons were recorded under voltage clamp during activation of astrocytic PAR1. A clear inward current (peak amplitude: 51±8.8 pA; n=6) was observed during activation of astrocytic PAR1 by bath application of 30 μM TFLLR, which is shown in FIG. 8d. That is, the activation of astrocytic PAR1 induces APV-sensitive inward current in PAR1−/− neurons. 30 μM TFLLR (bath application) increased intracellular Ca²⁺ level of wild-type astrocytes and simultaneously induced an inward current in adjacent PAR1−/− neurons (51±8.8 pA, n=6). This TFLLR-evoked current was blocked by 50 μM APV (peak amplitude: 2.5±0.3 pA, n=3), suggesting it reflects NMDA receptor activation. The current was abolished by the presence of 100 μM DL-APV (right panel).

Together these two results strongly suggest that the glutamate released by the astrocytes onto neurons in culture reach sufficient levels to activate NMDA receptors.

### Experimental Example 5

### Effect of Astrocytic PAR1 Activation on the Depolarization of Neurons in Slices

5.1: Examination of Glutamate Release in Hippocampal Slices

To determine whether PAR1-mediated glutamate release can occur in slices, whole cell patch recordings under voltage clamp from CA1 pyramidal cells during application of the PAR1 selective peptide agonist TFLLR (30 μM) or thrombin (30 nM) were obtained. After establishing a whole cell recording, slices were subsequently bathed in ACSF containing reduced Mg²⁺ (5 μM) supplemented with 0.5 μM tetrodotoxin (Tocris) for 15 minutes to allow measurement of the NMDA receptor current response. The obtained stable baseline suggests that this duration of low Mg²⁺ ACSF was sufficient to reduce extracellular Mg²⁺ to a stable level. The results are shown in FIGS. 9a and 9b.

FIG. 9 shows that PAR1 activation depolarizes neurons in hippocampal slices and increases membrane current noise, wherein FIG. 9a shows a representative trace showing 30 nM thrombin-induced inward current, which is abolished by switching to thrombin plus 100 μM APV. Recording was performed at −60 mV in the presence of 0.5 μM TTX and 5 μM Mg²⁺. FIG. 9b shows a summary of amplitude changes of inward current induced by thrombin and TFLLR with and without co-application of APV: thrombin: 8.8±3.1 pA, thrombin+APV: 2.4±1.3 pA; TFLLR: 15.9±3.1 pA, TFLLR+APV: 2.3±1.9 pA. * p<0.05, one-way repeated measure analysis of variance (Dunnett's method) compared with control.

As shown in FIGS. 9a and 9b, a clear inward current in response to thrombin (FIGS. 9a and 9b; n=8) or TFLLR (FIG. 9b; n=7) was observed, which was APV-sensitive for both PAR1 activators. A clear and significant increase in current fluctuations during the TFLLR or thrombin application in the presence of low external Mg²⁺ was observed. The noise increase was quantified by measuring the current variance in stretches of recordings with no spontaneous mEPSCs, and the results are shown in FIGS. 9c and 9d. FIG. 9c shows that the application of thrombin (30 nM) induces an increase in membrane current variance in the presence of TTX and the presence of low external Mg²⁺, which is blocked by 100 uM APV. mEPSCs were digitally removed as described in Example 5. FIG. 9d shows a summary of membrane current variance measurements from CA1 pyramidal cells held under voltage clamp (−60 mV) by thrombin (control (no treatment)): 7.7±1.0 pA²; thrombin: 12.4±1.1 pA²; thrombin+APV: 6.8±2.3 pA²) and the PAR1 agonist peptide TFLLR (30 μM; control: 6.1±0.7 pA²; TFLLR: 10.4±1.4 pA²; TFLLR+APV: 4.2±0.6 pA²); * p<0.05; one-way repeated measure analysis of variance (Dunnett's method) compared with control.

As shown in FIGS. 9c and 9d, the application of 30 μM TFLLR or 30 nM thrombin significantly increased the current variance, and this increase was reversed by the competitive NMDA receptor antagonist D-APV (50 μM).

This finding, coupled with the lack of any detectable PAR1 signaling in CA1 pyramidal cells (FIG. 3) and the lack of glutamate release from neurons (FIG. 7), strongly suggests that activation of astrocytic PAR1 stimulates release of glutamate that can in turn activate neuronal NMDA receptors in brain slices.

5.2: Examination of Neuron Depolarization Induced by PAR1 Induced Glutamate Release

In order to determine whether PAR1-evoked glutamate release was sufficient to depolarize neurons under normal conditions, the effect of 30 nM thrombin on membrane potential of CA1 pyramidal cells was evaluated in hippocampal slices bathed in normal ACSF (nominal 1.5 mM Mg²⁺). The results are shown in FIGS. 9e and 9f.

FIG. 9e shows the current clamp recording from a CA1 pyramidal cell (left panel) showing depolarization and spike firing during application of 30 nM thrombin (1.5 mM Mg²⁺). The inset shows spike firing during depolarizing current injection. The right panel is the summary of current clamp recordings from 22 neurons showing a significant depolarization of the membrane potential (p<0.01; paired t-test). As shown in FIG. 9e, a significant membrane depolarization (5.7±0.9 mV, range 0-15 mV, n=22, p<0.05, paired t-test; right panel) in current clamp recording from CA1 pyramidal neurons in hippocampal slices was observed.

FIG. 9f shows the decrease of depolarization by APV depending on the temperature, wherein APV (100 μM) significantly reduces thrombin-mediated depolarization of the membrane potential at 23° C. (thrombin: 5.8±1.7 mV; thrombin+APV: 2.5±1.0 mV, n=6) and at 34° C. (thrombin: 4.3±1.0 mV; thrombin+APV: 1.2±0.6 mV, n=12), * p<0.05; one-way repeated measure analysis of variance (Dunnett's method) compared with control. That is, additional current clamp recordings showed that application of APV (100 μM) significantly reduced the thrombin-induced depolarization. APV itself has no significant effect on the resting membrane potential of CA1 neurons (0.4±0.7 mV; n=4; p=0.32; paired t-test); the residual depolarization in APV was not significantly different than 0 mV (p>0.05; one-way repeated measure analysis of variance (Dunnett's method) compared with control). To verify that the APV-sensitive PAR1-mediated depolarization persisted in conditions of vigorous glutamate uptake, this experiment was repeated in slices held at 34° C. Identical results were found in that 30 nM thrombin induced a significant membrane depolarization that was significantly reduced by APV (n=12; FIG. 9f).

The APV sensitivity of the thrombin effect strongly supports the idea that PAR1-induced glutamate release from astrocytes depolarizes neurons through activation of NMDA receptors.

### Experimental Example 6

### Effect of Astrocytic PAR1-Mediated Glutamate Release on Synaptic NMDA Receptor Currents in Slices

6.1: Effect of PAR1 Activation on Decrease of Mg²⁺ Blockade of Synaptic NMDA Receptor

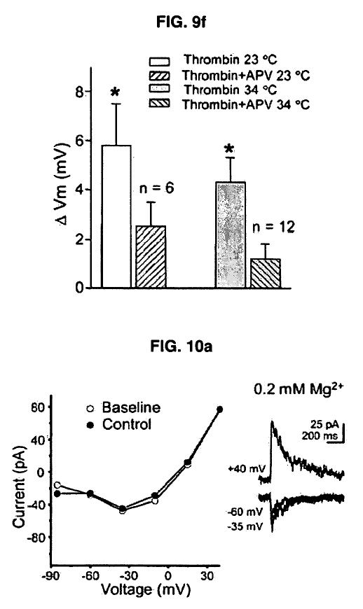

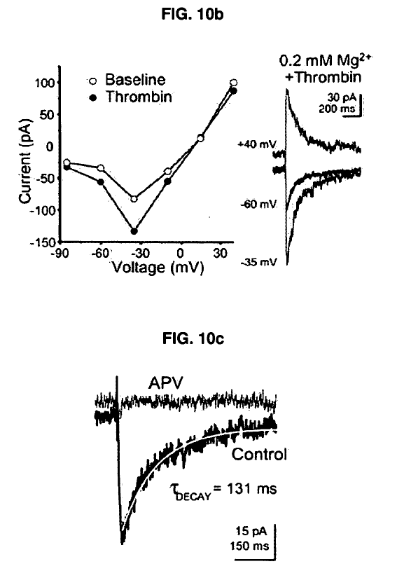

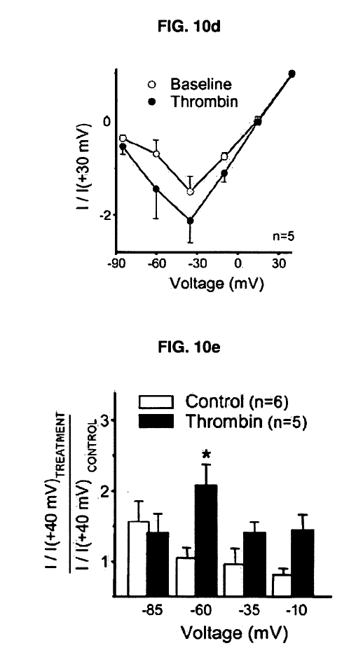

To test the idea that PAR1-stimulated glutamate release can depolarize distal dendrites in a manner that reduces Mg²⁺ block of synaptic NMDA receptors, the current-voltage (I-V) relationship for synaptically evoked excitatory postsynaptic currents (EPSCs) following PAR1 activation was examined. The results are shown in FIGS. 10a and 10b.

FIG. 10a shows the current voltage (I-V) relationship for evoked NMDA EPSCs recorded at 5 min intervals from CA1 pyramidal cells under voltage clamp at the indicated membrane potential. Slices were bathed in 10 μM CNQX and 20 μM bicuculline. External Mg²⁺ was reduced to 0.2 mM to allow visualization of the NMDA component. Control peak current is plotted as a function of holding potential. The inset shows the traces at the indicated holding potential. FIG. 10b shows a peak current by plotting as a function of membrane potential from a CA1 pyramidal cell before and 12.5 min following treatment with 30 nM thrombin. The right panel shows the traces at indicated holding potentials.

FIG. 10c shows the EPSCs recordings evoked from the case of being blocked by the competitive NMDA receptor antagonist D-APV (50 μM) and untreated control, confirming they were mediated by NMDA receptors. EPSCs were evoked by electrical stimulation of Schaffer collateral axons in the CA1 stratum radiatum, and recorded from CA1 pyramidal neurons under voltage clamp. External Mg²⁺ was reduced to 200 μM to allow measurement of NMDA receptor-mediated currents before PAR1 activation at hyperpolarized potentials where Mg²⁺ block is profound. All experiments were performed in the presence of 5 μM glycine, 20 μM CNQX and 20 μM bicuculline to isolate the NMDA component of the EPSCs, which decayed with a characteristically slow exponential time course (mean τ_{decay} 96 ms; n=6) that could be blocked by 100 μM APV (FIG. 10C).

FIG. 10d shows two I-V curves that are pooled before (2.5-12.5 min; open circles) and during (7.5-12.5 min; closed circles) treatment with 30 nM thrombin. The I-V curves were normalized to the current at +40 mV for each cell, and averaged across all cells. There was no change in membrane resistance (1.0 GΩ) or series resistance over the course of the experiment. FIG. 10e shows the relief of Mg²⁺ block for thrombin-treated cells compared to that for buffer-treated cells as the indicated ratio. *p<0.05, Mann-Whitney test.

Cells were held under voltage clamp at −60 mV, and the membrane potential stepped through 6 levels (+25 mV steps) at 30 sec intervals; I-V curves were collected every 5 min. After three baseline I-V curves were recorded, perfusion of the slice was switched to a reservoir containing either the same low Mg²⁺ artificial cerebrospinal fluid (ACSF) or low Mg²⁺ ACSF supplemented with 30 nM thrombin. The shape of the current-voltage relationship did not change in low Mg²⁺ ACSF-perfused control cells indicating no mechanical or perfusion artifact occurred with the solution switch (FIG. 10a). However, the NMDA receptor I-V curve was altered in thrombin-treated cells (FIG. 10b), with EPSCs showing significantly less Mg²⁺ block at a holding potential of −60 mV compared to control cells (FIGS. 10d and 10e). These results suggest that the PAR1 potentiation of NMDA receptor is most prominent at −60 mV, consistent with the idea that PAR1 activation causes a partial relief of Mg²⁺ blockade at this potential that is secondary to distal dendrite depolarization, which cannot be detected under voltage clamp. That is, as shown in FIGS. 10a-10e, it is revealed that the activation of PAR1 decreases Mg²⁺ blockade at synaptic NMDA receptors.

6.2: Effect of PAR1 Activation on Synaptic NMDA Receptor

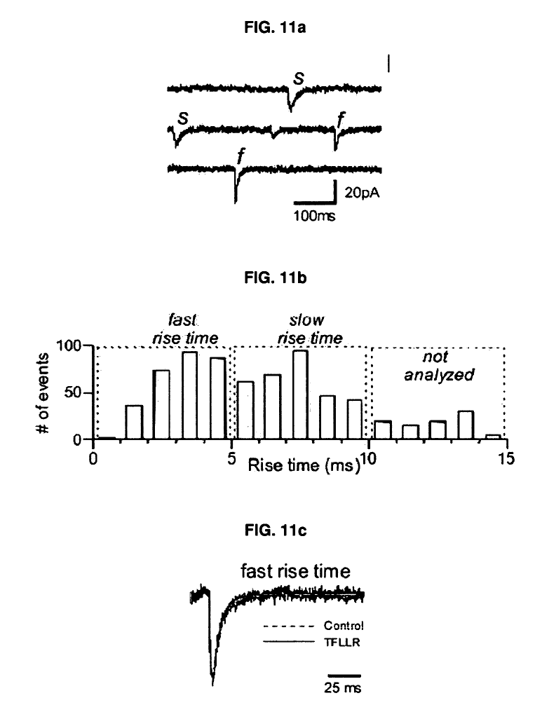

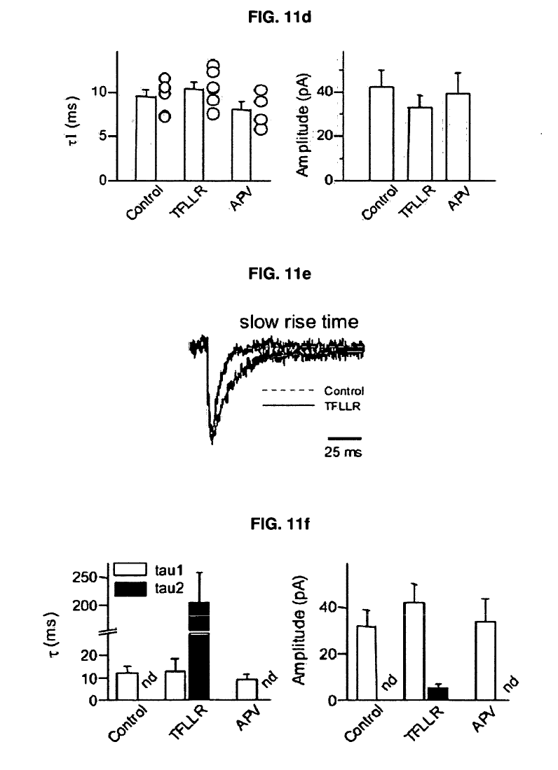

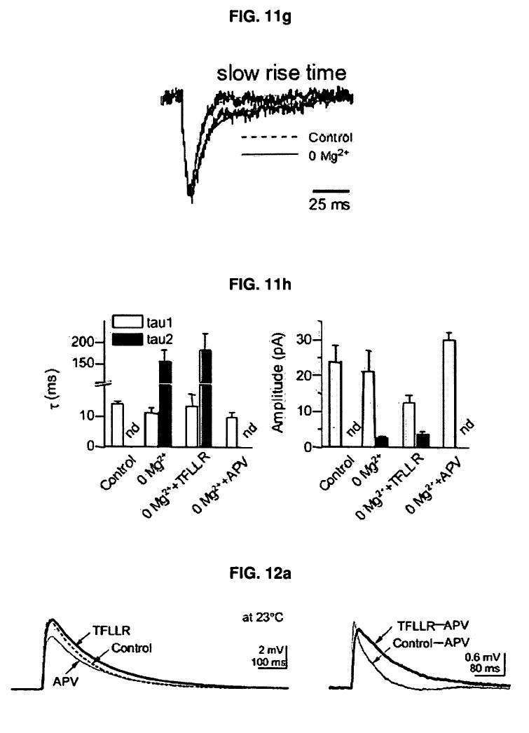

To investigate the effects of PAR1 activation on synaptic NMDA receptors in normal concentrations of Mg²⁺ (1.5 mM), the inventors recorded spontaneous miniature excitatory postsynaptic currents (mEPSCs) at −60 mV in the presence of 0.5 μM TTX to look for changes in the amplitude and decay kinetics of individual synaptic currents. The results are shown in FIGS. 11a to 11h.

FIG. 11a shows three selected traces demonstrating the different rise times of mEPSCs recorded under voltage clamp from CA1 pyramidal cells in hippocampal slices in 0.5 μM TTX (f and s stand for fast rise and slow rise mEPSCs, respectively). As shown in FIG. 11a, glutamatergic mEPSCs (frequency 0.1-0.5 Hz) recorded in the presence of 10 μM bicuculline and 1.5 mM extracellular Mg²⁺ showed the characteristic rapid rise and decay times, suggesting they arise primarily from AMPA receptor activation, with NMDA receptors subjected to strong voltage-dependent block by Mg²⁺. mEPSC rise time displayed a skewed distribution, which may be interpreted to reflect different electrotonic distances from the somatic recording site of synapses giving rise to mEPSCs (Rall, 1962; Stricker et al., 1996; Smith et al., 2003).

FIG. 11b is a histogram of the 10-90% rise times of 692 mEPSCs recorded from II cells for 5 min. Two subgroups of mEPSCs were selected for analysis based on the rise time, as shown in the rise time distribution. The first group had a rise time of 1-5 ms, whereas the second group showed a slow rise time (5-10 ms). Inward currents with rise times slower than 10 ms were rare, likely reflected poorly clamped EPSCs subject to heavy electrotonic filtering, and thus were not analyzed.

FIG. 11c shows superimposed normalized average traces showing fast rising mEPSCs in the absence and presence of TFLLR. The average traces were best fitted with a single component exponential function, shown in green for control and red for TFLLR, which superimpose. FIG. 11d is a bar graph showing no significant difference in the decay time constant τ₁ (left panel) or amplitude (right panel) of fast rising mEPSCs recorded under control conditions or during application of TFLLR or APV (50 μM). No significant differences were observed in both of decay time constant and amplitude.

FIG. 11e shows superimposed normalized average traces showing slow rising mEPSCs from the same cell as in (11d) in the absence and presence of TFLLR. The average traces were best fitted with a single component exponential function (control, green) or a two component exponential function (TFLLR, red). It was revealed that the activation of PAR1 by TFLLR induced the appearance of a slowly decaying synaptic current. FIG. 11f is a bar graph showing no significant difference in the decay time constant τ₁ (left panel) or amplitude (right panel) of fastest component of mEPSCs recorded under all conditions. By contrast, a second slower decay time constant (τ₂=205±54 ms, n=6) was evident only after TFLLR treatment. APV eliminated TFLLR-induced τ₂; “nd” indicates that τ₂ was not detected.

Application of 30 μM TFLLR to selectively activate PAR1 had no significant effect on the time course or amplitude of mEPSCs with a faster rise time (<5 ms), assumed to arise from more proximal synapses under relatively good voltage control (FIGS. 11c and 11d; p>0.05; paired t-test). By contrast, application of TFLLR markedly prolonged the decay of the average time course for slow rising mEPSCs recorded in the presence of extracellular Mg²⁺ compared to control mEPSCs; there was no significant change in the frequency or peak amplitude (FIG. 11e). The prolonged time course was manifested as an appearance of a second slow decay time constant in the presence of TFLLR (FIG. 11f). This TFLLR-dependent slow component of the mEPSC decay was sensitive to the NMDA receptor blocker APV (50 μM; FIG. 11f), whereas the average peak amplitude and frequency of mEPSCs were unaffected by APV. Interestingly, the increase in decay time was only observed in the mEPSCs with a slower rise time (>5 ms), which we interpret to represent quantal events arising at distal synapses at which the voltage clamp is less effective, thus allowing PAR I-mediated depolarization.

6.3: Role of Extracellular Mg²⁺ on PAR1 Potentiation of NMDA Receptor Function

The inventors subsequently tested the role of extracellular Mg²⁺ on PAR1 potentiation of NMDA receptor function by removing Mg²⁺ from the extracellular solution. The results are shown in FIGS. 11g and 11h. FIG. 11g shows superimposed normalized average traces showing that slow rising mEPSCs possessed a slow NMDA receptor mediated component in nominal absence of extracellular Mg²⁺ (0 Mg²⁺). As shown in FIG. 11g, under this condition, control mEPSCs showed a prominent and slow NMDA receptor mediated synaptic component. FIG. 11h shows that TFLLR had no significant effect on fitted time constants or amplitudes of the two synaptic components in Mg²⁺ free ACSF. APV eliminated the slowest component in the absence of Mg²⁺, confirming that it was mediated by NMDA receptors.

As shown in FIGS. 11g and 11h, application of TFLLR had no significant effect on either the slow decay time constant or the fitted amplitude of the NMDA component in the absence of Mg²⁺ (p>0.05, paired t-test). These data indicate that extracellular Mg²⁺ is required for the PAR1 potentiation of postsynaptic NMDA receptors. This increase of the NMDA receptor component of distal but not proximal mEPSCs along with the Mg²⁺-dependence suggest that PAR1-induced potentiation of NMDA receptors involves depolarization of the distal dendrites that in turn causes a relief of the synaptic Mg²⁺ block. If extracellular Mg²⁺ is removed and there is no voltage-dependent block of NMDA receptors, than the distal depolarization will not lead to any potentiation of the unblocked NMDA receptors.

### Experimental Example 7

### Effects of Astrocytic PAR1-Mediated Glutamate Release on Synaptic NMDA Receptor Potentials

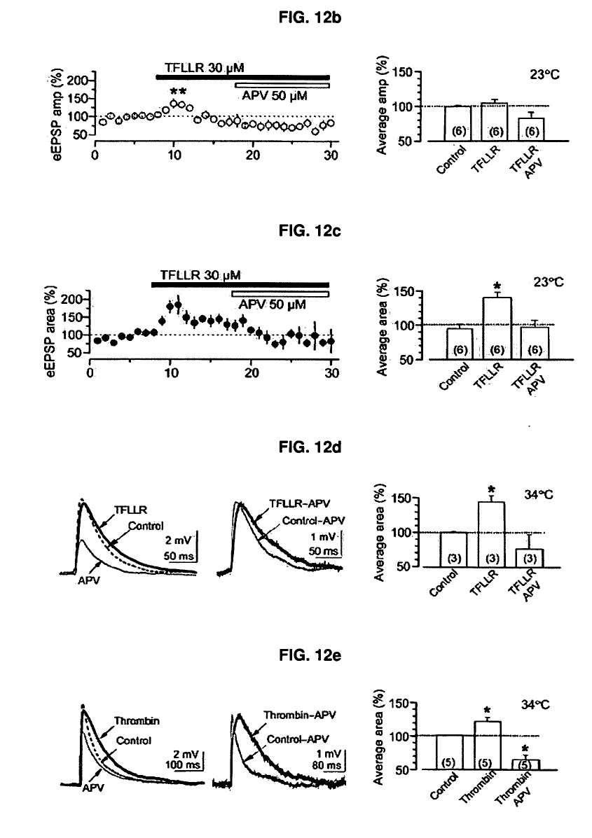

A series of current clamp experiments were carried out to directly measure the effects of PAR1 activation on the NMDA receptor-mediated component of the EPSP recorded in the presence of 1.5 mM Mg²⁺. In this example, QX314 (Sigma) was included in the intracellular pipette solution to block action potentials in the recorded neuron, and also included 10 μM bicuculline in the extracellular solution and a surgical cut was performed between CA3 and CA1 regions to prevent polysynaptic feedback inhibition and excitation from contaminating the EPSP waveform. The results are shown in FIGS. 12a to 12e.

In FIG. 12a, the left panel shows average EPSP obtained from a single representative rat CA1 pyramidal cell before (blue), during application of the PAR1 agonist TFLLR (black, 30 μM), and in the presence of APV (grey, 50 μM). The right panel shows the difference potential between the EPSP recorded under control and APV, or following TFLLR and APV. The enhancement of the late phase of the EPSP by PAR1 activation was observed. As shown in FIG. 12a, application of a 0.1 ms 1-100 μA stimulus to the Schaffer collaterals with a bipolar electrode evoked a monosynaptic EPSPs that rapidly rose to a mean amplitude of 3.7±0.9 mV (FIG. 12a; n=6). The EPSP decayed with a slow time course (˜tau decay 65 ms), consistent with it being a dual AMPA and NMDA receptor-mediated EPSP, as expected following the reduction of feedback inhibition (Collingridge et al., 1988).

FIG. 12b shows an average time course of the peak amplitude during application of 30 μM TFLLR; error bars are SEM. The right panel shows TFLLR activation of PAR1 has no significant effect on the overall amplitude of the EPSP (p>0.05) although there is a significant difference on the time points marked by asterisks on the left panel; number in parentheses is the number of cells. When 30 30M TFLLR was applied to the slices, the late phase of the EPSP appeared to be potentiated, as shown by comparison of the difference potential between EPSPs recorded under control conditions and APV or TFLLR and APV. There was no significant effect on the amplitude of the EPSP after a modest and transient potentiation (asterisk in FIG. 12b).

FIG. 12c shows an average time course of the area under the EPSP during application of the PAR1 activator TFLLR (30 μM) and APV (100 μM). The right panel shows TFLLR significantly enhances the area of the EPSP, consistent with an enhancement of the NMDA component. The enhancement is blocked by 100 μM APV; * p<0.05, one-way repeated measure analysis of variance (Dunnett's method) compared with control. In contrast to FIG. 12b, TFLLR-mediated PAR1 activation significantly increased the integral (i.e. area) of the EPSP to 140±8% of control (n=6; p<0.05). Application of 50 μM APV reversed this potentiation by TFLLR at the conclusion of the experiment.

To confirm that the astrocytic release of glutamate that appears to depolarize neurons at 23° C. also occurs under more physiological conditions with robust glutamate uptake, the present inventors repeated the experiment in slices at 34° C. The results are shown in FIGS. 12d and 12e. FIGS. 12d and 12e show the potentiation of synaptic NMDA component of EPSPs by PAR1 activation (30 μM TFLLR by 145±9%, and 30 nM thrombin: 120±6%) near physiologic temperature (34° C.); * p<0.05, one-way repeated measure analysis of variance (Dunnett's method) compared with control. These results confirm that both activators of thrombin are effective at enhancing the NMDA component of EPSPs, and that the effects occur under near physiological conditions (34° C.).

In conclusion, FIGS. 12a to 12e show that PAR1 activation potentiates the synaptic NMDA component of EPSPs in CA1 pyramidal neurons.

By the above examples, the mechanism, wherein activation of astrocytic PAR, especially PAR1, specifically affects postsynaptic neurons, thereby inducing glutamate receptor activation and depolarization in the same direction with neural transmission, is revealed. Therefore, the present invention is useful in developing technologies relating to neuro-protection and stimulation of neurotransmission through controlling astrocytic PARs.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A controlling method of activity of a glutamate receptor on a postsynaptic neuron, comprising the steps of:
(1) controlling a G-protein coupled receptor on an astrocyte to control glutamate release through the astrocytic G-protein coupled receptor; and
(2) controlling activity of the glutamate receptor on an adjacent postsynaptic neuron by the released glutamate.

2. The method according to claim 1, wherein the G-protein coupled receptor on the astrocyte is one or more selected from the group consisting of P2Y receptors, bradykinin receptors, and protease activated receptors (PARs), and the glutamate receptor on the adjacent postsynaptic neuron is a N-methyl-D-aspartic acid (NMDA) receptor.

3. The method according to claim 1, wherein the controlling method is to activate the activity of the glutamate receptor on a postsynaptic neuron by activating the G-protein coupled receptor and stimulating glutamate release.

4. The method according to claim 3, further comprising the step of stimulating glutamate receptor mediated neurotransmission by the activation of the glutamate receptor.

5. The method according to claim 1, wherein the controlling method is to inactivate the activity of the glutamate receptor on a postsynaptic neuron by inactivating the G-protein coupled receptor and inhibiting glutamate release.

6. The method according to claim 5, further comprising the step of protecting nerve cells from glutamate neurotoxicity by the inactivation of the glutamate receptor.

7. A method of screening a neuroprotecting agent, comprising the steps of:
contacting a candidate compound selectively with a G-protein coupled receptor on an astrocyte;
measuring inward current through an N-methyl-D-aspartic acid (NMDA) receptor on an adjacent postsynaptic neuron; and
determining the candidate compound as a neuroprotecting agent that protects neural cells from neurotoxicity caused by over-release of glutamate from an astrocyte and over-activation of an NMDA receptor of an adjacent postsynaptic neuron, when the inward current in the case of contacting the candidate compound is decreased compared with that of the case of not contacting the candidate compound.

8. The method according to claim 7, wherein the G-protein coupled receptor on an astrocyte is one or more selected from the group consisting of P2Y receptors, bradykinin receptors, and protease activated receptors (PARs).

9. The method according to claim 7, wherein the neuroprotecting agent has a protecting, improving, or treating effect against one or more brain diseases caused by glutamate neurotoxicity selected from epileptic seizures caused by over-release of glutamate during a seizure, ischemia, stroke, cerebral hemorrhage, epilepsy, brain injury, and hypoxia.
